(19) United States
(12) Patent Application Publication
(10) Pub. No.: US 2026/0335319 A1
(43) Pub. Date: Oct. 29, 2026

(54) INJECTABLE COMPOSITIONS OF METOLAZONE

(71) Applicant: CIPLA LIMITED, Mumbai (IN)

(72) Inventors: Pradeep BHADAURIA, Weston, FL (US); Arunprasath KALIAPERUMAL, Weston, FL (US); Tushar RAJYAGURU, Bhavnagar (IN); Harshad HARDE, Nashik (IN); Sandip KHAIRNAR, Nashik (IN)

(21) Appl. No.: 19/689,550

(22) Filed: May 27, 2026

Related U.S. Application Data
(63) Continuation of application No. 19/478,803, filed on Apr. 25, 2024.

(30) Foreign Application Priority Data
Apr. 27, 2023 (IN) 202321030280

(51) Int. Cl. A61K 31/517; A61K 9/00; A61K 47/10; A61K 47/34; A61K 47/44

(52) U.S. Cl. CPC A61K 31/517; A61K 9/0019; A61K 47/10; A61K 47/34; A61K 47/44

(57) ABSTRACT

The present invention relates to injectable pharmaceutical composition comprising metolazone. The invention particularly relates to a ready-to-dilute composition comprising metolazone and pharmaceutically acceptable excipients, process of preparing the composition and its use in the treatment of salt and water retention.

### CROSS-REFERENCE TO RELATED APPLICATIONS

This application is a U.S. continuation application of U.S. application Ser. No. 19/478,803, filed on Oct. 25, 2025 which is a U.S. national phase application, filed under 35 U.S.C. § 371, of International Patent Application No. PCT/IN2024/050443, filed on Apr. 25, 2024, which claims the benefit of Indian Patent Application number 202321030280, filed Apr. 27, 2023, the contents of which are incorporated by reference herein in their entirety.

### FIELD OF INVENTION

The present invention relates to an injectable pharmaceutical composition comprising metolazone. The invention particularly relates to a ready-to-dilute composition comprising metolazone, and pharmaceutically acceptable excipients The invention also particularly relates to a process of preparing the composition and its use in the treatment of salt and water retention.

### BACKGROUND OF INVENTION

Metolazone is a poorly aqueous soluble drug that has poor bioavailability and variable absorption. Metolazone is only sparingly soluble in water, but more soluble in alkali, and organic solvents. Solubility and stability are long identified factors hindering the development of therapeutic agents. Conventional delivery systems for very insoluble drugs typically use a combination of specialized excipients like cyclodextrins, extreme pH conditions, all of which are detrimental and toxic, when ingested in large amounts.

Metolazone is marketed as an oral immediate release tablet under the trademark Zaroxolyn™ in the United States. However, these metolazone oral tablet formulations are known to have a variable bioavailability and absorption which is erratic. This problem is particularly seen in patients with severe gastrointestinal edema. This gastrointestinal edema eventually causes structural and functional abnormalities of the gastrointestinal tract, which in turn results in reduction of rate of drug absorption and further delays the time to achieve an effective therapeutic dose. Further, such oral dosage forms cannot be administered in patients who are not conscious and have difficulty in swallowing (dysphagia). Hence there remains a need in the art for an injectable dosage form of metolazone, which can overcome the variable and erratic absorption and bioavailability issues observed with oral formulations. Further, it would be convenient to administer such injectable dosage forms to patients who are not conscious and who have difficulty in swallowing.

Metolazone has limited solubility in aqueous solutions and formulation of an injectable composition is very complex. It is challenging to formulate an injectable composition of metolazone wherein the metolazone is in dissolved state and precipitation is prevented throughout the shelf of the product. Further, such injectable composition needs to be diluted in compatible admixture diluents before administration. Owing to the low aqueous solubility of metolazone, there is a high chance of precipitation of metolazone when diluted with compatible admixture diluents. It is of utmost challenge to prevent precipitation of metolazone at this stage, and this makes the formulation further challenging.

There have been some prior attempts on formulation of metolazone in an injectable composition, however, there is limited data on stability of such injectable composition upon dilution with compatible admixture diluents. Thus, there exists a need in the art to develop a ready-to-dilute composition comprising metolazone.

### OBJECT OF THE INVENTION

An object of the invention is to provide an injectable composition comprising metolazone.

Another object of the invention is to provide an injectable composition comprising (a) metolazone, (b) at least one solvent, (c) at least one surfactant, and (d) a pharmaceutically acceptable vehicle.

Another object of the invention is to provide a process for preparing stable injectable composition comprising metolazone.

One another object of the present invention is to provide safe, efficacious, and ready to dilute injectable composition comprising metolazone.

Another object of the present invention is to provide a method of treating salt and water retention by administering a ready-to-dilute injectable composition comprising metolazone.

### SUMMARY OF THE INVENTION

The present invention relates to injectable pharmaceutical composition comprising metolazone. The invention particularly relates to a ready-to-dilute composition comprising metolazone, and pharmaceutically acceptable excipients, process of preparing the composition, and its use in the treatment of salt and water retention. Prior attempts on formulation of metolazone in an injectable composition have not led to a stable composition and especially such formulation were seen to precipitate upon dilution with compatible admixture diluents. Thus, there exists a need in the art to develop a ready-to-dilute composition comprising metolazone. The present invention addresses the unmet need and provides an injectable composition of metolazone which can be administered to a patient in need thereof. The injectable composition of the present invention also exhibits sufficient stability and prevents precipitation of metolazone even after dilution with compatible admixture diluents.

The present invention relates to an injectable composition of the present invention comprising (a) metolazone, (b) at least one solvent, (c) at least one surfactant, and (d) a pharmaceutically acceptable vehicle.

In one embodiment, the injectable composition of the present invention comprises metolazone in an amount of about 0.01 mg to about 50 mg.

In a further embodiment, the injectable composition of the present invention comprises metolazone in an amount of about 0.01 mg to about 20 mg.

In another embodiment, the injectable composition of the present invention comprises metolazone in an amount of about 0.1 mg to about 10 mg.

In another embodiment, the injectable composition of the present invention comprises metolazone in an amount of about 1 mg to about 10 mg.

In one more embodiment, the injectable composition of the present invention comprises metolazone an amount of about 5 mg.

In one embodiment, the injectable composition of the present invention comprises metolazone in the unionized form.

In one embodiment, the injectable composition of the present invention comprises metolazone as a pharmaceutically acceptable salt thereof.

In one embodiment, the injectable composition of the present invention comprises solvent in an amount of about 1 mg to about 1500 mg.

In one embodiment, the injectable composition of the present invention comprises solvent in an amount of about 1 mg to about 700 mg.

In a further embodiment, the injectable composition of the present invention comprises solvent in an amount of about 100 mg to about 600 mg.

In one more embodiment, the injectable composition of the present invention comprises solvent in an amount of about 200 mg.

In another embodiment, the injectable composition of the present invention comprises solvent in an amount of about 300 mg.

In an embodiment, the injectable composition of the present invention comprises solvent in an amount of about 400 mg.

In one more embodiment, the injectable composition of the present invention comprises solvent in an amount of about 800 mg.

In another embodiment, the injectable composition of the present invention comprises solvent in an amount of about 1000 mg.

In one embodiment, the injectable composition of the present invention comprises solvent in an amount of about 0.1% w/v to about 70% w/v.

In one more embodiment, the injectable composition of the present invention comprises solvent in an amount of about 10% w/v to about 60% w/v.

In a further embodiment, the injectable composition of the present invention comprises solvent in an amount of about 20% w/v.

In one more embodiment, the injectable composition of the present invention comprises solvent in an amount of about 30% w/v.

In one embodiment, the injectable composition of the present invention comprises solvent in an amount of about 37.5% w/v.

In another embodiment, the injectable composition of the present invention comprises solvent in an amount of about 40% w/v.

In a further embodiment, the injectable composition of the present invention comprises solvent in an amount of about 50% w/v.

In one embodiment, the injectable composition of the present invention comprises solvent in an amount of about 62.5% w/v.

In a further embodiment, the injectable composition of the present invention comprises surfactant in an amount of about 1 mg to about 500 mg.

In one more embodiment, the injectable composition of the present invention comprises surfactant is present in an amount of about 100 mg.

In an embodiment, the injectable composition of the present invention comprises surfactant is present in an amount of about 125 mg.

In one embodiment, the injectable composition of the present invention comprises surfactant is present in an amount of about 150 mg.

In a further embodiment, the injectable composition of the present invention comprises surfactant is present in an amount of about 175 mg.

In one more embodiment, the injectable composition of the present invention comprises surfactant in an amount of about 200 mg.

In another embodiment, the injectable composition of the present invention comprises surfactant in an amount of about 300 mg.

In a further embodiment, the injectable composition of the present invention comprises surfactant in an amount of about 400 mg.

In one embodiment, the injectable composition of the present invention comprises surfactant in an amount of about 0.1% w/v to about 50% w/v.

In another embodiment, the injectable composition of the present invention comprises surfactant in an amount of about 6.25% w/v.

In one more embodiment, the injectable composition of the present invention comprises surfactant in an amount of about 10% w/v.

In an embodiment, the injectable composition of the present invention comprises surfactant in an amount of about 12.5% w/v.

In one embodiment, the injectable composition of the present invention comprises surfactant in an amount of about 15% w/v.

In a further embodiment, the injectable composition of the present invention comprises surfactant in an amount of about 17.5% w/v.

In an embodiment, the injectable composition of the present invention comprises surfactant in an amount of about 18.75% w/v.

In one more embodiment, the injectable composition of the present invention comprises surfactant in an amount of about 20% w/v.

In one more embodiment, the injectable composition of the present invention comprises surfactant in an amount of about 25% w/v.

In a further embodiment, the injectable composition of the present invention comprises surfactant in an amount of about 30% w/v.

In one embodiment, the injectable composition of the present invention comprises (a) about 0.01 mg to about 20 mg of metolazone, (b) about 1 mg to about 700 mg of at least one solvent, (c) about 1 mg to about 500 mg of at least one surfactant, and (d) a pharmaceutically acceptable vehicle.

In a further embodiment, the injectable composition of the present invention comprises (a) about 0.01 mg to about 20 mg of metolazone, (b) about 100 mg to about 600 mg of at least one solvent, (c) about 1 mg to about 500 mg of at least one surfactant, and (d) a pharmaceutically acceptable vehicle.

In one more embodiment, the injectable composition of the present invention comprises (a) about 0.01 mg to about 20 mg of metolazone, (b) about 100 mg to about 600 mg of at least one solvent, (c) about 100 mg of at least one surfactant, and (d) a pharmaceutically acceptable vehicle.

In one embodiment, the injectable composition of the present invention comprises (a) about 0.01 mg to about 20 mg of metolazone, (b) about 100 mg to about 600 mg of at least one solvent, (c) about 125 mg of at least one surfactant, and (d) a pharmaceutically acceptable vehicle.

In another embodiment, the injectable composition of the present invention comprises (a) about 0.01 mg to about 20 mg of metolazone, (b) about 100 mg to about 600 mg of at least one solvent, (c) about 150 mg of at least one surfactant, and (d) a pharmaceutically acceptable vehicle.

In a further embodiment, the injectable composition of the present invention comprises (a) about 0.01 mg to about 20 mg of metolazone, (b) about 100 mg to about 600 mg of at least one solvent, (c) about 175 mg of at least one surfactant, and (d) a pharmaceutically acceptable vehicle.

In another embodiment, the injectable composition of the present invention comprises (a) about 0.01 mg to about 20 mg of metolazone, (b) about 100 mg to about 600 mg of at least one solvent, (c) about 200 mg of at least one surfactant, and (d) a pharmaceutically acceptable vehicle.

In one another embodiment, the injectable composition of the present invention comprises (a) about 0.01 mg to about 20 mg of metolazone, (b) about 100 mg to about 600 mg of at least one solvent, (c) about 300 mg of at least one surfactant, and (d) a pharmaceutically acceptable vehicle.

In a further embodiment, the injectable composition of the present invention comprises (a) about 5 mg of metolazone, (b) about 200 mg of at least one solvent, (c) about 100 mg of at least one surfactant, and (d) a pharmaceutically acceptable vehicle.

In an embodiment, the injectable composition of the present invention comprises (a) about 5 mg of metolazone, (b) about 200 mg of at least one solvent, (c) about 125 mg of at least one surfactant, and (d) a pharmaceutically acceptable vehicle.

In one embodiment, the injectable composition of the present invention comprises (a) about 5 mg of metolazone, (b) about 200 mg of at least one solvent, (c) about 150 mg of at least one surfactant, and (d) a pharmaceutically acceptable vehicle.

In another embodiment, the injectable composition of the present invention comprises (a) about 5 mg of metolazone, (b) about 200 mg of at least one solvent, (c) about 175 mg of at least one surfactant, and (d) a pharmaceutically acceptable vehicle.

In an embodiment, the injectable composition of the present invention comprises (a) about 5 mg of metolazone, (b) about 200 mg of at least one solvent, (c) about 200 mg of at least one surfactant, and (d) a pharmaceutically acceptable vehicle.

In a further embodiment, the injectable composition of the present invention comprises (a) about 5 mg of metolazone, (b) about 200 mg of at least one solvent, (c) about 300 mg of at least one surfactant, and (d) a pharmaceutically acceptable vehicle.

In one more embodiment, the injectable composition of the present invention comprises (a) about 5 mg of metolazone, (b) about 300 mg of at least one solvent, (c) about 100 mg of at least one surfactant, and (d) a pharmaceutically acceptable vehicle.

In one embodiment, the injectable composition of the present invention comprises (a) about 5 mg of metolazone, (b) about 300 mg of at least one solvent, (c) about 125 mg of at least one surfactant, and (d) a pharmaceutically acceptable vehicle.

In an embodiment, the injectable composition of the present invention comprises (a) about 5 mg of metolazone, (b) about 300 mg of at least one solvent, (c) about 150 mg of at least one surfactant, and (d) a pharmaceutically acceptable vehicle.

In one more embodiment, the injectable composition of the present invention comprises (a) about 5 mg of metolazone, (b) about 300 mg of at least one solvent, (c) about 175 mg of at least one surfactant, and (d) a pharmaceutically acceptable vehicle.

In one embodiment, the injectable composition of the present invention comprises (a) about 5 mg of metolazone, (b) about 300 mg of at least one solvent, (c) about 200 mg of at least one surfactant, and (d) a pharmaceutically acceptable vehicle.

In one more embodiment, the injectable composition of the present invention comprises (a) about 5 mg of metolazone, (b) about 300 mg of at least one solvent, (c) about 300 mg of at least one surfactant, and (d) a pharmaceutically acceptable vehicle.

In an embodiment, the injectable composition of the present invention comprises (a) about 5 mg of metolazone, (b) about 400 mg of at least one solvent, (c) about 100 mg of at least one surfactant, and (d) a pharmaceutically acceptable vehicle.

In one more embodiment, the injectable composition of the present invention comprises (a) about 5 mg of metolazone, (b) about 400 mg of at least one solvent, (c) about 125 mg of at least one surfactant, and (d) a pharmaceutically acceptable vehicle.

In another embodiment, the injectable composition of the present invention comprises (a) about 5 mg of metolazone, (b) about 400 mg of at least one solvent, (c) about 150 mg of at least one surfactant, and (d) a pharmaceutically acceptable vehicle.

In an embodiment, the injectable composition of the present invention comprises (a) about 5 mg of metolazone, (b) about 400 mg of at least one solvent, (c) about 175 mg of at least one surfactant, and (d) a pharmaceutically acceptable vehicle.

In one embodiment, the injectable composition of the present invention comprises (a) about 5 mg of metolazone, (b) about 400 mg of at least one solvent, (c) about 200 mg of at least one surfactant, and (d) a pharmaceutically acceptable vehicle.

In an embodiment, the injectable composition of the present invention comprises (a) about 5 mg of metolazone, (b) about 400 mg of at least one solvent, (c) about 300 mg of at least one surfactant, and (d) a pharmaceutically acceptable vehicle.

In one embodiment, the injectable composition of the present invention comprises (a) about 5 mg of metolazone, (b) about 1000 mg of solvent, (c) about 100 mg of surfactant, and (d) a pharmaceutically acceptable vehicle. In one more embodiment, the composition has a volume of about 1.6 ml.

In one more embodiment, the injectable composition of the present invention comprises (a) about 5 mg of metolazone, (b) about 800 mg of solvent, (c) about 300 mg of surfactant, and (d) a pharmaceutically acceptable vehicle. In one more embodiment, the composition has a volume of about 1.6 ml.

In a further embodiment, the injectable composition of the present invention comprises (a) about 5 mg of metolazone, (b) about 600 mg of solvent, (c) about 400 mg of surfactant, and (d) a pharmaceutically acceptable vehicle. In one embodiment, the composition has a volume of about 2 ml.

In one embodiment, the injectable composition of the present invention comprises (a) about 0.01 mg to about 20 mg of metolazone, (b) about 0.1% w/v to about 70% w/v of at least one solvent, (c) about 0.1% w/v to about 50% w/v of at least one surfactant, and (d) a pharmaceutically acceptable vehicle.

In a further embodiment, the injectable composition of the present invention comprises (a) about 0.01 mg to about 20 mg of metolazone, (b) about 10% w/v to about 60% w/v of at least one solvent, (c) about 0.1% w/v to about 50% w/v of at least one surfactant, and (d) a pharmaceutically acceptable vehicle.

In one more embodiment, the injectable composition of the present invention comprises (a) about 0.01 mg to about 20 mg of metolazone, (b) about 10% w/v to about 60% w/v of at least one solvent, (c) about 10% w/v of at least one surfactant, and (d) a pharmaceutically acceptable vehicle.

In one embodiment, the injectable composition of the present invention comprises (a) about 0.01 mg to about 20 mg of metolazone, (b) about 10% w/v to about 60% w/v of at least one solvent, (c) about 12.5% w/v of at least one surfactant, and (d) a pharmaceutically acceptable vehicle.

In one another embodiment, the injectable composition of the present invention comprises (a) about 0.01 mg to about 20 mg of metolazone, (b) about 10% w/v to about 60% w/v of at least one solvent, (c) about 15% w/v of at least one surfactant, and (d) a pharmaceutically acceptable vehicle.

In one more embodiment, the injectable composition of the present invention comprises (a) about 0.01 mg to about 20 mg of metolazone, (b) about 10% w/v to about 60% w/v of at least one solvent, (c) about 17.5% w/v of at least one surfactant, and (d) a pharmaceutically acceptable vehicle.

In another embodiment, the injectable composition of the present invention comprises (a) about 0.01 mg to about 20 mg of metolazone, (b) about 10% w/v to about 60% w/v of at least one solvent, (c) about 20% w/v of at least one surfactant, and (d) a pharmaceutically acceptable vehicle.

In an embodiment, the injectable composition of the present invention comprises (a) about 0.01 mg to about 20 mg of metolazone, (b) about 10% w/v to about 60% w/v of at least one solvent, (c) about 30% w/v of at least one surfactant, and (d) a pharmaceutically acceptable vehicle.

In one embodiment, the injectable composition of the present invention comprises (a) about 5 mg of metolazone, (b) about 20% w/v of at least one solvent, (c) about 10% w/v of at least one surfactant, and (d) a pharmaceutically acceptable vehicle.

In one more embodiment, the injectable composition of the present invention comprises (a) about 5 mg of metolazone, (b) about 20% w/v of at least one solvent, (c) about 12.5% w/v of at least one surfactant, and (d) a pharmaceutically acceptable vehicle.

In one another embodiment, the injectable composition of the present invention comprises (a) about 5 mg of metolazone, (b) about 20% w/v of at least one solvent, (c) about 15% w/v of at least one surfactant, and (d) a pharmaceutically acceptable vehicle.

In an embodiment, the injectable composition of the present invention comprises (a) about 5 mg of metolazone, (b) about 20% w/v of at least one solvent, (c) about 17.5% w/v of at least one surfactant, and (d) a pharmaceutically acceptable vehicle.

In a further embodiment, the injectable composition of the present invention comprises (a) about 5 mg of metolazone, (b) about 20% w/v of at least one solvent, (c) about 20% w/v of at least one surfactant, and (d) a pharmaceutically acceptable vehicle.

In another embodiment, the injectable composition of the present invention comprises (a) about 5 mg of metolazone, (b) about 20% w/v of at least one solvent, (c) about 30% w/v of at least one surfactant, and (d) a pharmaceutically acceptable vehicle.

In a further embodiment, the injectable composition of the present invention comprises (a) about 5 mg of metolazone, (b) about 30% w/v of at least one solvent, (c) about 10% w/v of at least one surfactant, and (d) a pharmaceutically acceptable vehicle.

In an embodiment, the injectable composition of the present invention comprises (a) about 5 mg of metolazone, (b) about 30% w/v of at least one solvent, (c) about 12.5% w/v of at least one surfactant, and (d) a pharmaceutically acceptable vehicle.

In a further embodiment, the injectable composition of the present invention comprises (a) about 5 mg of metolazone, (b) about 30% w/v of at least one solvent, (c) about 15% w/v of at least one surfactant, and (d) a pharmaceutically acceptable vehicle.

In an embodiment, the injectable composition of the present invention comprises (a) about 5 mg of metolazone, (b) about 30% w/v of at least one solvent, (c) about 17.5% w/v of at least one surfactant, and (d) a pharmaceutically acceptable vehicle.

In one embodiment, the injectable composition of the present invention comprises (a) about 5 mg of metolazone, (b) about 30% w/v of at least one solvent, (c) about 20% w/v of at least one surfactant, and (d) a pharmaceutically acceptable vehicle.

In another embodiment, the injectable composition of the present invention comprises (a) about 5 mg of metolazone, (b) about 30% w/v of at least one solvent, (c) about 30% w/v of at least one surfactant, and (d) a pharmaceutically acceptable vehicle.

In a further embodiment, the injectable composition of the present invention comprises (a) about 5 mg of metolazone, (b) about 40% w/v of at least one solvent, (c) about 10% w/v of at least one surfactant, and (d) a pharmaceutically acceptable vehicle.

In an embodiment, the injectable composition of the present invention comprises (a) about 5 mg of metolazone, (b) about 40% w/v of at least one solvent, (c) about 12.5% w/v of at least one surfactant, and (d) a pharmaceutically acceptable vehicle.

In a further embodiment, the injectable composition of the present invention comprises (a) about 5 mg of metolazone, (b) about 40% w/v of at least one solvent, (c) about 15% w/v of at least one surfactant, and (d) a pharmaceutically acceptable vehicle.

In another embodiment, the injectable composition of the present invention comprises (a) about 5 mg of metolazone, (b) about 40% w/v of at least one solvent, (c) about 17.5% w/v of at least one surfactant, and (d) a pharmaceutically acceptable vehicle.

In one embodiment, the injectable composition of the present invention comprises (a) about 5 mg of metolazone, (b) about 40% w/v of at least one solvent, (c) about 20% w/v of at least one surfactant, and (d) a pharmaceutically acceptable vehicle.

In another embodiment, the injectable composition of the present invention comprises (a) about 5 mg of metolazone, (b) about 40% w/v of at least one solvent, (c) about 30% w/v of at least one surfactant, and (d) a pharmaceutically acceptable vehicle.

In one embodiment, the injectable composition of the present invention comprises solvent comprising at least one of polyethylene glycol, propylene glycol, and ethanol.

In another embodiment, the injectable composition of the present invention comprises polysorbate as a surfactant.

In one more embodiment, the injectable composition of the present invention comprises PEG 35 Castor oil as a surfactant.

In one embodiment, the injectable composition of the present invention comprises water as a pharmaceutically acceptable vehicle.

In one embodiment, the injectable composition of the present invention comprises (a) about 0.1 mg to about 10 mg of metolazone, (b) about 0.1% w/v to about 70% w/v of at least one solvent, (c) about 0.1% w/v to about 50% w/v of at least one surfactant, and (d) a pharmaceutically acceptable vehicle.

In another embodiment, the injectable composition of the present invention comprises (a) about 0.1 mg to about 10 mg of metolazone, (b) about 30% w/v to about 65% w/v of at least one solvent, (c) about 5% w/v to about 30% w/v of at least one surfactant, and (d) a pharmaceutically acceptable vehicle.

In one more embodiment, the injectable composition of the present invention comprises (a) about 0.1 mg to about 10 mg of metolazone, (b) about 0.1% w/v to about 70% w/v of ethanol, (c) about 0.1% w/v to about 50% w/v of PEG 35 castor oil, and (d) a pharmaceutically acceptable vehicle.

In a further embodiment, the injectable composition of the present invention comprises (a) about 0.1 mg to about 10 mg of metolazone, (b) about 30% w/v to about 65% w/v of ethanol, (c) about 5% w/v to about 30% w/v of PEG 35 castor oil, and (d) a pharmaceutically acceptable vehicle.

In an embodiment, the injectable composition of the present invention comprises (a) about 0.1 mg to about 10 mg of metolazone, (b) about 62.5% w/v of ethanol, (c) about 6.25% w/v of PEG 35 castor oil, and (d) water.

In one more embodiment, the injectable composition of the present invention comprises (a) about 0.1 mg to about 10 mg of metolazone, (b) about 50% w/v of ethanol, (c) about 18.75% w/v of PEG 35 castor oil, and (d) water.

In a further embodiment, the injectable composition of the present invention comprises (a) about 0.1 mg to about 10 mg of metolazone, (b) about 30% w/v of ethanol, (c) about 20% w/v of PEG 35 castor oil, and (d) water.

In one embodiment, the injectable composition of the present invention comprises (a) about 5 mg of metolazone, (b) about 62.5% w/v of solvent, (c) about 6.25% w/v of surfactant, and (d) a pharmaceutically acceptable vehicle.

In one more embodiment, the injectable composition of the present invention comprises (a) about 5 mg of metolazone, (b) about 50% w/v of solvent, (c) about 18.75% w/v of surfactant, and (d) a pharmaceutically acceptable vehicle.

In a further embodiment, the injectable composition of the present invention comprises (a) about 5 mg of metolazone, (b) about 30% w/v of solvent, (c) about 20% w/v of surfactant, and (d) a pharmaceutically acceptable vehicle.

In one more embodiment, the injectable composition of the present invention is stable for at least 1 hour after dilution with a compatible admixture diluent.

In an embodiment, the injectable composition of the present invention is an aqueous composition.

Also disclosed herein are processes for preparation of an injectable composition comprising metolazone. Also disclosed herein are methods for treating salt and water retention by administering an injectable composition comprising metolazone.

Other objects, features and advantages of the present invention will be apparent to those of ordinary skill in the art in view of the following detailed description of the invention and accompanying drawings.

### DETAILED DESCRIPTION OF THE INVENTION

The present invention relates to stable injectable pharmaceutical composition comprising metolazone. The invention particularly relates to a ready-to-dilute composition comprising metolazone and pharmaceutically acceptable excipients, process of preparing the composition and its use in the treatment of salt and water retention.

Metolazone is designated chemically as 7-chloro-1, 2, 3,4-tetrahydro-2-methyl-3-(2-methylphenyl)-4-oxo-6-quinazolinesulfonamide and a molecular weight of 365.83. Metolazone can exist in the unionized form, or as a pharmaceutically acceptable salt. The chemical structure in the unionized form is:

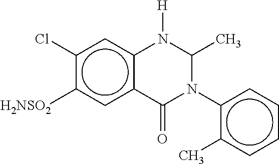

Metolazone has limited solubility in aqueous solutions and formulation of an injectable composition is complex. Metolazone is available as an oral dosage form viz. immediate release tablet, which is reported to have erratic absorption and variable bioavailability. There remains a need in the art for an injectable dosage form of metolazone, which can overcome the variable and erratic absorption and bioavailability issues observed with oral formulations. Further, it would be convenient to administer such injectable dosage forms to patients who are not conscious and who have difficulty in swallowing.

Owing to the limited aqueous solubility, it is challenging to formulate an injectable composition of metolazone wherein the metolazone is in a dissolved state and precipitation is prevented throughout the shelf life of the product. Further, such injectable composition are typically diluted in compatible admixture diluents before administration. Owing to the low aqueous solubility of metolazone, there is a high chance of precipitation of metolazone when diluted with compatible admixture diluents. It is of utmost challenge to prevent precipitation of metolazone at this stage, and this makes the formulation further challenging. Thus, there exists a need in the art to develop a ready-to-dilute composition comprising metolazone.

The present invention addresses this unmet need and provides an injectable composition of metolazone, which can be administered to a patient in need thereof. The injectable composition of the present invention also exhibits sufficient stability and prevents precipitation of metolazone even after dilution with compatible admixture diluents. The inventors of the present invention through rigorous experimentation have found out that a combination of surfactants and solvents imparts sufficient stability to the injectable composition of metolazone, and prevents precipitation of metolazone even after dilution with compatible admixture diluents.

The injectable composition of the present invention comprises metolazone, along with excipients. The injectable composition of the present invention may include pharmaceutically acceptable salts of metolazone such as, sodium, maleate, sulphonate, succinate and hydrochloride. The particle size is a critical attribute for improving the solubilization of metolazone. The particle size of metolazone as per the present invention may have a D90 about 10 μm to about 200 μm, preferably from about 15 μm to about 100 μm. In some embodiments, the particle size of metolazone may have a D90 of at least about 10 μm 20 μm., 30 μm, 40 μm, 50 μm, 60 μm. In some embodiments, the composition contains at least about 0.01 mg to about 50 mg metolazone, preferably about 0.01 mg to about 20 mg metolazone, preferably about 0.1 mg to about 10 mg, more preferably about 5 mg. The injectable composition of the present invention comprises about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg metolazone or its pharmaceutically acceptable salt thereof. Preferably, the injectable composition of the present invention comprises about 1 mg, about 2.5 mg, about 5 mg, about 7.5 mg or about 10 mg metolazone.

The following description sets forth exemplary embodiments of the present technology. It should be recognized, however, that such description is not intended as a limitation on the scope of the present disclosure but is instead provided as a description of exemplary embodiments.

As used in the present specification, the following words, phrases, and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X.” Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The injectable composition of the present invention comprises (a) metolazone, (b) at least one solvent, (c) at least one surfactant, and (d) a pharmaceutically acceptable vehicle. In another embodiment, the injectable composition of the present invention is aqueous.

The injectable composition of the present invention comprises a combination of surfactants and solvents to allow for solubilization of metolazone as well as impart stability to the composition after dilution with compatible admixture diluents for infusion.

The injectable composition of the present invention comprises at least one solvent. Non-limiting examples of solvent are ethanol, pentane, 2-methylbutane (isopentane), heptane, hexane, cyclopentane and cyclohexane, methanol, 2-methoxyethanol, isopropanol, n-butanol, t-butyl alcohol, octanol, ethyl acetate, 2-methoxyethyl acetate, butyl acetate, benzyl benzoate, benzene, toluene, pyridine, xylene, diethyl ether, 2-ethoxyethyl ether, ethylene glycol dimethyl ether, methyl t-butyl ether, formaldehyde, glutaraldehyde, acetone, 3-pentanone (diethyl ketone), ethylene glycol, propylene glycol, formic acid, acetic acid, trifluoroacetic acid, phosphoric acid, acetic anhydride, piperidine, N,N-dimethylacetamide, N,N-dimethylformamide, dimethylsulfoxide (DMSO), benzonitrile, acetonitrile, hydrazine, dichloromethane, chloroform, carbon tetrachloride, 1,2-dichlorobenzene, 1,2-dichloroethane, tetrachloroethylene and 1-chlorobutane, tetrahydrofuran,1,4-dioxane; glycerin/glycerol, Polyethylene glycol (PEG) e.g.: PEG 400 and Polypropylene glycol (PPG), e.g. PPG 400, PPG 1200 and PPG 2000.

In one more embodiment, the injectable composition of the present invention comprises a solvent comprising at least one of ethanol, propylene glycol, and polyethylene glycol.

In an embodiment, the injectable composition of the present invention comprises solvent in an amount of about 1 mg to about 1500 mg.

In one embodiment, the injectable composition of the present invention comprises solvent in an amount of about 1 mg to about 700 mg.

In a further embodiment, the injectable composition of the present invention comprises solvent in an amount of about 100 mg to about 600 mg.

In an embodiment, the injectable composition of the present invention comprises solvent in an amount of about 200 mg.

In one more embodiment, the injectable composition of the present invention comprises solvent in an amount of about 300 mg.

In another embodiment, the injectable composition of the present invention comprises solvent in an amount of about 400 mg.

In one more embodiment, the injectable composition of the present invention comprises solvent in an amount of about 800 mg.

In other embodiment, the injectable composition of the present invention comprises solvent in an amount of about 1000 mg.

In one embodiment, the injectable composition of the present invention comprises solvent in an amount of about 0.1% w/v to about 70% w/v.

In one more embodiment, the injectable composition of the present invention comprises solvent in an amount of about 10% w/v to about 60% w/v.

In an embodiment, the injectable composition of the present invention comprises solvent in an amount of about 20% w/v.

In one embodiment, the injectable composition of the present invention comprises solvent in an amount of about 30% w/v.

In one embodiment, the injectable composition of the present invention comprises solvent in an amount of about 37.5% w/v.

In another embodiment, the injectable composition of the present invention comprises solvent in an amount of about 40% w/v.

In a further embodiment, the injectable composition of the present invention comprises solvent in an amount of about 50% w/v.

In one embodiment, the injectable composition of the present invention comprises solvent in an amount of about 62.5% w/v.

The injectable composition of the present invention comprises at least one surfactant. The term “surfactant” as used herein means agents that lower the surface tension (or interfacial tension) between two liquids or between a liquid and a solid. Surfactants are usually organic compounds that are amphiphilic, i.e., they contain both hydrophobic groups (tails) and hydrophilic groups (heads). Therefore, a surfactant contains both a water-insoluble (or oil-soluble) component and a water-soluble component. Surfactants can be classified according to polar head group. A non-ionic surfactant has no charged groups in its head. Non-ionic surfactants have covalently bonded oxygen-containing hydrophilic groups, which are bonded to hydrophobic parent structures. The head of an ionic surfactant carries a net positive, or negative charge. If the charge is negative, the surfactant is more specifically called anionic; if the charge is positive, it is called cationic. If a surfactant contains a head with two oppositely charged groups, it is termed zwitterionic. An ideal interaction of hydrophilic and lipophilic group of surfactant with particle surface could be reason for obtaining appropriate suspension stability. The surfactant can be a non-ionic surfactant, an ionic surfactant (anionic or cationic), or a zwitterionic surfactant. The surfactant reduces the interfacial tension between the solid particles and the liquid vehicle. Pharmaceutically acceptable surfactant for this application include, but are not limited to, D-α-Tocopheryl polyethylene glycol 1000 succinate (Vitamin E TPGS), polysorbate or polyethoxylated castor oil, Polyoxyl 20 stearate, Polyoxyl 35 castor oil, poloxamer, polyoxyethylene sorbitan monoisostearate, polyethylene glycol 40 sorbitan diisostearate, Polyoxyl 40 Hydrogenated castor oil, Polysorbate, Polysorbate 20, Polysorbate 40, Polyoxyl 60 stearate, Polysorbate 85, Polysorbate 60, poloxamer 331, polyoxyethylene fatty acid esters, Polyoxyl 40 castor oil, poloxamer 188, polyoxyethylene polyoxypropylene 1800, oleic acid, Sodium desoxycholate, Sodium lauryl sulfate, Sorbitan monolaurate, Sorbitan monooleate, Sorbitan monopalmitate, Sorbitan trioleate, N-Carbamoyl methoxypolyethylene glycol 2000-1,2-distearol, myristic acid, Steareth, Stearic acid, Polyoxyl 40 stearate, Sucrose stearate, Tocopherol, polyoxyl castor oil, Triglyceride synthetic, Trimyristin, Tristearin, magnesium stearate, lecithin, lauryl sulfate, Vitamin E, egg yolk phosphatides, docusate sodium, Polysorbate 80, dimyristoyl phosphatidylglycerol, dimyristoyl lecithin, Capryol 90 (propylene glycol monocaprylate), Capryol PGMC (propylene glycol monocaprylate), deoxycholate, cholesterol, Cremophor EL, Propylene glycol alginate, Croval A-10 (PEG 60 almond glycerides), Labrafil 1944 (oleoyl macrogol-6 glycerides), Labrafil 2125 (linoleoyl macrogol-6 glycerides), Labrasol (caprylocaproyl macrogol-8 glycerides), Lauroglycol 90 (propylene glycol monolaurate), Lauroglycol FCC (propylene glycol laurate), calcium stearate, Lecithin Centromix E, Lecithin Centrophase 152, Lecithin Centrol 3F21B, POE 26 glycerin, Olepal isosteariques (PEG-6 isostearate), Plurol diisostearique (polyglycerol-3-diisostearate), Plurol Oleique CC, POE 20 Sorbitan trioleate, Tagat TO (polyoxyethylene glycerol trioleate), or Solutol (macrogol-15 hydroxystearate). Preferred surfactants include Polysorbate 20, Polysorbate 80, sorbitan monolaurate, and poloxamer 188.

In an embodiment, the injectable composition of the present invention comprises a surfactant having an HLB value greater than about 8. In one embodiment, the surfactant has an HLB value of about 10 to about 30.

In a further embodiment, the injectable composition of the present invention comprises surfactant in an amount of about 1 mg to about 500 mg.

In one embodiment, the injectable composition of the present invention comprises surfactant is present in an amount of about 100 mg.

In an embodiment, the injectable composition of the present invention comprises surfactant is present in an amount of about 125 mg.

In one embodiment, the injectable composition of the present invention comprises surfactant is present in an amount of about 150 mg.

In a further embodiment, the injectable composition of the present invention comprises surfactant is present in an amount of about 175 mg.

In one more embodiment, the injectable composition of the present invention comprises surfactant in an amount of about 200 mg.

In another embodiment, the injectable composition of the present invention comprises surfactant in an amount of about 300 mg.

In a further embodiment, the injectable composition of the present invention comprises surfactant in an amount of about 400 mg.

In one embodiment, the injectable composition of the present invention comprises surfactant in an amount of about 0.1% w/v to about 50% w/v.

In another embodiment, the injectable composition of the present invention comprises surfactant in an amount of about 6.25% w/v.

In one more embodiment, the injectable composition of the present invention comprises surfactant in an amount of about 10% w/v.

In an embodiment, the injectable composition of the present invention comprises surfactant is present in an amount of about 12.5% w/v.

In one embodiment, the injectable composition of the present invention comprises surfactant is present in an amount of about 15% w/v.

In a further embodiment, the injectable composition of the present invention comprises surfactant is present in an amount of about 17.5% w/v.

In an embodiment, the injectable composition of the present invention comprises surfactant is present in an amount of about 18.75% w/v.

In one more embodiment, the injectable composition of the present invention comprises surfactant in an amount of about 20% w/v.

In one more embodiment, the injectable composition of the present invention comprises surfactant in an amount of about 25% w/v.

In a further embodiment, the injectable composition of the present invention comprises surfactant is present in an amount of about 30% w/v.

In one embodiment, the injectable composition of the present invention comprises solvent in an amount of 30% w/v, 30.25% w/v, 30.5% w/v, 30.75% w/v, 31% w/v, 31.25% w/v, 31.5% w/v, 31.75% w/v, 32% w/v, 32.25% w/v, 32.5% w/v, 32.75% w/v, 33% w/v, 33.25% w/v, 33.5% w/v, 33.75% w/v, 34% w/v, 34.25% w/v, 34.5% w/v, 34.75% w/v, 35% w/v, 35.25% w/v, 35.5% w/v, 35.75% w/v, 36% w/v, 36.25% w/v, 36.5% w/v, 36.75% w/v, 37% w/v, 37.25% w/v, 37.5% w/v, 37.75% w/v, 38% w/v, 38.25% w/v, 38.5% w/v, 38.75% w/v, 39% w/v, 39.25% w/v, 39.5% w/v, 39.75% w/v, 40% w/v, 40.25% w/v, 40.5% w/v, 40.75% w/v, 41% w/v, 41.25% w/v, 41.5% w/v, 41.75% w/v, 42% w/v, 42.25% w/v, 42.5% w/v, 42.75% w/v, 43% w/v, 43.25% w/v, 43.5% w/v, 43.75% w/v, 44% w/v, 44.25% w/v, 44.5% w/v, 44.75% w/v, 45% w/v, 45.25% w/v, 45.5% w/v, 45.75% w/v, 46% w/v, 46.25% w/v, 46.5% w/v, 46.75% w/v, 47% w/v, 47.25% w/v, 47.5% w/v, 47.75% w/v, 48% w/v, 48.25% w/v, 48.5% w/v, 48.75% w/v, 49% w/v, 49.25% w/v, 49.5% w/v, 49.75% w/v, 50% w/v, 50.25% w/v, 50.5% w/v, 50.75% w/v, 51% w/v, 51.25% w/v, 51.5% w/v, 51.75% w/v, 52% w/v, 52.25% w/v, 52.5% w/v, 52.75% w/v, 53% w/v, 53.25% w/v, 53.5% w/v, 53.75% w/v, 54% w/v, 54.25% w/v, 54.5% w/v, 54.75% w/v, 55% w/v, 55.25% w/v, 55.5% w/v, 55.75% w/v, 56% w/v, 56.25% w/v, 56.5% w/v, 56.75% w/v, 57% w/v, 57.25% w/v, 57.5% w/v, 57.75% w/v, 58% w/v, 58.25% w/v, 58.5% w/v, 58.75% w/v, 59% w/v, 59.25% w/v, 59.5% w/v, 59.75% w/v, 60% w/v, 60.25% w/v, 60.5% w/v, 60.75% w/v, 61% w/v, 61.25% w/v, 61.5% w/v, 61.75% w/v, 62% w/v, 62.25% w/v, 62.5% w/v, 62.75% w/v, 63% w/v, 63.25% w/v, 63.5% w/v, 63.75% w/v, 64% w/v, 64.25% w/v, 64.5% w/v, 64.75% w/v, or 65% w/v.

In an embodiment, the injectable composition of the present invention comprises ethanol in an amount of 30% w/v, 30.25% w/v, 30.5% w/v, 30.75% w/v, 31% w/v, 31.25% w/v, 31.5% w/v, 31.75% w/v, 32% w/v, 32.25% w/v, 32.5% w/v, 32.75% w/v, 33% w/v, 33.25% w/v, 33.5% w/v, 33.75% w/v, 34% w/v, 34.25% w/v, 34.5% w/v, 34.75% w/v, 35% w/v, 35.25% w/v, 35.5% w/v, 35.75% w/v, 36% w/v, 36.25% w/v, 36.5% w/v, 36.75% w/v, 37% w/v, 37.25% w/V, 37.5% w/v, 37.75% w/v, 38% w/v, 38.25% w/v, 38.5% w/v, 38.75% w/v, 39% w/v, 39.25% w/v, 39.5% w/v, 39.75% w/v, 40% w/v, 40.25% w/v, 40.5% w/v, 40.75% w/v, 41% w/v, 41.25% w/v, 41.5% w/v, 41.75% w/v, 42% w/v, 42.25% w/v, 42.5% w/v, 42.75% w/v, 43% w/v, 43.25% w/v, 43.5% w/v, 43.75% w/v, 44% w/v, 44.25% w/v, 44.5% w/v, 44.75% w/v, 45% w/v, 45.25% w/v, 45.5% w/v, 45.75% w/v, 46% w/v, 46.25% w/v, 46.5% w/v, 46.75% w/v, 47% w/v, 47.25% w/v, 47.5% w/v, 47.75% w/v, 48% w/v, 48.25% w/v, 48.5% w/v, 48.75% w/v, 49% w/v, 49.25% w/v, 49.5% w/v, 49.75% w/v, 50% w/v, 50.25% w/v, 50.5% w/v, 50.75% w/v, 51% w/v, 51.25% w/v, 51.5% w/v, 51.75% w/v, 52% w/v, 52.25% w/v, 52.5% w/v, 52.75% w/v, 53% w/v, 53.25% w/v, 53.5% w/v, 53.75% w/v, 54% w/v, 54.25% w/v, 54.5% w/v, 54.75% w/v, 55% w/v, 55.25% w/v, 55.5% w/v, 55.75% w/v, 56% w/v, 56.25% w/v, 56.5% w/v, 56.75% w/v, 57% w/v, 57.25% w/v, 57.5% w/v, 57.75% w/v, 58% w/v, 58.25% w/v, 58.5% w/v, 58.75% w/v, 59% w/v, 59.25% w/v, 59.5% w/v, 59.75% w/v, 60% w/v, 60.25% w/v, 60.5% w/v, 60.75% w/v, 61% w/v, 61.25% w/v, 61.5% w/v, 61.75% w/v, 62% w/v, 62.25% w/v, 62.5% w/v, 62.75% w/v, 63% w/v, 63.25% w/v, 63.5% w/v, 63.75% w/v, 64% w/v, 64.25% w/v, 64.5% w/v, 64.75% w/v, or 65% w/v.

In one embodiment, the injectable composition of the present invention comprises surfactant in an amount of 5% w/v, 5.25% w/v, 5.5% w/v, 5.75% w/v, 6% w/v, 6.25% w/v, 6.5% w/v, 6.75% w/v, 7% w/v, 7.25% w/v, 7.5% w/v, 7.75% w/v, 8% w/v, 8.25% w/v, 8.5% w/v, 8.75% w/v, 9% w/v, 9.25% w/v, 9.5% w/v, 9.75% w/v, 10% w/v, 10.25% w/v, 10.5% w/v, 10.75% w/V, 11% w/v, 11.25% w/v, 11.5% w/v, 11.75% w/v, 12% w/v, 12.25% w/v, 12.5% w/v, 12.75% w/v, 13% w/v, 13.25% w/v, 13.5% w/v, 13.75% w/v, 14% w/v, 14.25% w/v, 14.5% w/v, 14.75% w/v, 15% w/v, 15.25% w/v, 15.5% w/v, 15.75% w/v, 16% w/v, 16.25% w/v, 16.5% w/v, 16.75% w/V, 17% w/v, 17.25% w/v, 17.5% w/v, 17.75% w/v, 18% w/v, 18.25% w/v, 18.5% w/v, 18.75% w/v, 19% w/v, 19.25% w/v, 19.5% w/v, 19.75% w/v, 20% w/v, 20.25% w/v, 20.5% w/v, 20.75% w/v, 21% w/v, 21.25% w/v, 21.5% w/v, 21.75% w/v, 22% w/v, 22.25% w/v, 22.5% w/v, 22.75% w/v, 23% w/v, 23.25% w/v, 23.5% w/v, 23.75% w/v, 24% w/v, 24.25% w/v, 24.5% w/v, 24.75% w/V, 25% w/v, 25.25% w/v, 25.5% w/v, 25.75% w/v, 26% w/v, 26.25% w/v, 26.5% w/v, 26.75% w/v, 27% w/v, 27.25% w/v, 27.5% w/v, 27.75% w/v, 28% w/v, 28.25% w/v, 28.5% w/v, 28.75% w/v, 29% w/v, 29.25% w/v, 29.5% w/v, 29.75% w/v, or 30% w/v.

In one more embodiment, the injectable composition of the present invention comprises PEG 35 castor oil in an amount of 5% w/v, 5.25% w/v, 5.5% w/v, 5.75% w/v, 6% w/v, 6.25% w/v, 6.5% w/v, 6.75% w/v, 7% w/v, 7.25% w/v, 7.5% w/v, 7.75% w/v, 8% w/v, 8.25% w/v, 8.5% w/v, 8.75% w/v, 9% w/v, 9.25% w/v, 9.5% w/v, 9.75% w/v, 10% w/v, 10.25% w/v, 10.5% w/v, 10.75% w/v, 11% w/v, 11.25% w/v, 11.5% w/v, 11.75% w/v, 12% w/v, 12.25% w/v, 12.5% w/V, 12.75% w/v, 13% w/v, 13.25% w/v, 13.5% w/v, 13.75% w/v, 14% w/v, 14.25% w/v, 14.5% w/v, 14.75% w/v, 15% w/v, 15.25% w/v, 15.5% w/v, 15.75% w/v, 16% w/v, 16.25% w/v, 16.5% w/v, 16.75% w/v, 17% w/v, 17.25% w/v, 17.5% w/v, 17.75% w/v, 18% w/v, 18.25% w/v, 18.5% w/v, 18.75% w/v, 19% w/v, 19.25% w/v, 19.5% w/v, 19.75% w/v, 20% w/v, 20.25% w/v, 20.5% w/v, 20.75% w/v, 21% w/v, 21.25% w/v, 21.5% w/v, 21.75% w/v, 22% w/v, 22.25% w/v, 22.5% w/v, 22.75% w/v, 23% w/v, 23.25% w/v, 23.5% w/v, 23.75% w/v, 24% w/v, 24.25% w/v, 24.5% w/v, 24.75% w/v, 25% w/v, 25.25% w/v, 25.5% w/v, 25.75% w/v, 26% w/v, 26.25% w/v, 26.5% w/v, 26.75% w/v, 27% w/v, 27.25% w/v, 27.5% w/v, 27.75% w/v, 28% w/v, 28.25% w/v, 28.5% w/v, 28.75% w/v, 29% w/v, 29.25% w/v, 29.5% w/v, 29.75% w/v, or 30% w/v.

The injectable composition of the present invention may also contain a pharmaceutically acceptable vehicle, which allows for solubilization of the active and excipients and also aids in stabilization of the composition. In an embodiment, the injectable composition of the present invention contains water as a pharmaceutically acceptable vehicle.

The present invention relates to an injectable composition of the present invention comprising (a) metolazone, (b) at least one solvent, (c) at least one surfactant, and (d) a pharmaceutically acceptable vehicle.

In one embodiment, the injectable composition of the present invention comprises solvent comprising at least one of polyethylene glycol, propylene glycol, and ethanol.

In another embodiment, the injectable composition of the present invention comprises a surfactant which is a non-ionic surfactant.

In one more embodiment, the injectable composition of the present invention comprises polysorbate as a surfactant.

In another embodiment, the injectable composition of the present invention comprises PEG 35 Castor oil as a surfactant.

In one embodiment, the injectable composition of the present invention comprises metolazone in an amount of about 0.01 mg to about 50 mg.

In a further embodiment, the injectable composition of the present invention comprises metolazone in an amount of about 0.01 mg to about 20 mg.

In another embodiment, the injectable composition of the present invention comprises metolazone in an amount of about 1 mg to about 10 mg.

In an embodiment, the injectable composition of the present invention comprises metolazone an amount of about 5 mg.

In one embodiment, the injectable composition of the present invention comprises (a) about 0.01 mg to about 20 mg of metolazone, (b) about 1 mg to about 700 mg of at least one solvent, (c) about 1 mg to about 500 mg of at least one surfactant and (d) a pharmaceutically acceptable vehicle.

In a further embodiment, the injectable composition of the present invention comprises (a) about 0.01 mg to about 20 mg of metolazone, (b) about 100 mg to about 600 mg of at least one solvent, (c) about 1 mg to about 500 mg of at least one surfactant and (d) a pharmaceutically acceptable vehicle.

In one more embodiment, the injectable composition of the present invention comprises (a) about 0.01 mg to about 20 mg of metolazone, (b) about 100 mg to about 600 mg of at least one solvent, (c) about 100 mg of at least one surfactant and (d) a pharmaceutically acceptable vehicle.

In one embodiment, the injectable composition of the present invention comprises (a) about 0.01 mg to about 20 mg of metolazone, (b) about 100 mg to about 600 mg of at least one solvent, (c) about 125 mg of at least one surfactant and (d) a pharmaceutically acceptable vehicle.

In another embodiment, the injectable composition of the present invention comprises (a) about 0.01 mg to about 20 mg of metolazone, (b) about 100 mg to about 600 mg of at least one solvent, (c) about 150 mg of at least one surfactant and (d) a pharmaceutically acceptable vehicle.

In a further embodiment, the injectable composition of the present invention comprises (a) about 0.01 mg to about 20 mg of metolazone, (b) about 100 mg to about 600 mg of at least one solvent, (c) about 175 mg of at least one surfactant and (d) a pharmaceutically acceptable vehicle.

In another embodiment, the injectable composition of the present invention comprises (a) about 0.01 mg to about 20 mg of metolazone, (b) about 100 mg to about 600 mg of at least one solvent, (c) about 200 mg of at least one surfactant and (d) a pharmaceutically acceptable vehicle.

In one another embodiment, the injectable composition of the present invention comprises (a) about 0.01 mg to about 20 mg of metolazone, (b) about 100 mg to about 600 mg of at least one solvent, (c) about 300 mg of at least one surfactant and (d) a pharmaceutically acceptable vehicle.

In a further embodiment, the injectable composition of the present invention comprises (a) about 5 mg of metolazone, (b) about 200 mg of at least one solvent, (c) about 100 mg of at least one surfactant and (d) a pharmaceutically acceptable vehicle.

In an embodiment, the injectable composition of the present invention comprises (a) about 5 mg of metolazone, (b) about 200 mg of at least one solvent, (c) about 125 mg of at least one surfactant and (d) a pharmaceutically acceptable vehicle.

In one embodiment, the injectable composition of the present invention comprises (a) about 5 mg of metolazone, (b) about 200 mg of at least one solvent, (c) about 150 mg of at least one surfactant and (d) a pharmaceutically acceptable vehicle.

In another embodiment, the injectable composition of the present invention comprises (a) about 5 mg of metolazone, (b) about 200 mg of at least one solvent, (c) about 175 mg of at least one surfactant and (d) a pharmaceutically acceptable vehicle.

In one embodiment, the injectable composition of the present invention comprises (a) about 5 mg of metolazone, (b) about 200 mg of at least one solvent, (c) about 200 mg of at least one surfactant and (d) a pharmaceutically acceptable vehicle.

In an embodiment, the injectable composition of the present invention comprises (a) about 5 mg of metolazone, (b) about 200 mg of at least one solvent, (c) about 300 mg of at least one surfactant and (d) a pharmaceutically acceptable vehicle.

In a further embodiment, the injectable composition of the present invention comprises (a) about 5 mg of metolazone, (b) about 300 mg of at least one solvent, (c) about 100 mg of at least one surfactant and (d) a pharmaceutically acceptable vehicle.

In one embodiment, the injectable composition of the present invention comprises (a) about 5 mg of metolazone, (b) about 300 mg of at least one solvent, (c) about 125 mg of at least one surfactant and (d) a pharmaceutically acceptable vehicle.

In an embodiment, the injectable composition of the present invention comprises (a) about 5 mg of metolazone, (b) about 300 mg of at least one solvent, (c) about 150 mg of at least one surfactant and (d) a pharmaceutically acceptable vehicle.

In one more embodiment, the injectable composition of the present invention comprises (a) about 5 mg of metolazone, (b) about 300 mg of at least one solvent, (c) about 175 mg of at least one surfactant and (d) a pharmaceutically acceptable vehicle.

In one embodiment, the injectable composition of the present invention comprises (a) about 5 mg of metolazone, (b) about 300 mg of at least one solvent, (c) about 200 mg of at least one surfactant and (d) a pharmaceutically acceptable vehicle.

In an embodiment, the injectable composition of the present invention comprises (a) about 5 mg of metolazone, (b) about 300 mg of at least one solvent, (c) about 300 mg of at least one surfactant and (d) a pharmaceutically acceptable vehicle.

In a further embodiment, the injectable composition of the present invention comprises (a) about 5 mg of metolazone, (b) about 400 mg of at least one solvent, (c) about 100 mg of at least one surfactant and (d) a pharmaceutically acceptable vehicle.

In one more embodiment, the injectable composition of the present invention comprises (a) about 5 mg of metolazone, (b) about 400 mg of at least one solvent, (c) about 125 mg of at least one surfactant and (d) a pharmaceutically acceptable vehicle.

In another embodiment, the injectable composition of the present invention comprises (a) about 5 mg of metolazone, (b) about 400 mg of at least one solvent, (c) about 150 mg of at least one surfactant and (d) a pharmaceutically acceptable vehicle.

In an embodiment, the injectable composition of the present invention comprises (a) about 5 mg of metolazone, (b) about 400 mg of at least one solvent, (c) about 175 mg of at least one surfactant and (d) a pharmaceutically acceptable vehicle.

In one embodiment, the injectable composition of the present invention comprises (a) about 5 mg of metolazone, (b) about 400 mg of at least one solvent, (c) about 200 mg of at least one surfactant and (d) a pharmaceutically acceptable vehicle.

In an embodiment, the injectable composition of the present invention comprises (a) about 5 mg of metolazone, (b) about 400 mg of at least one solvent, (c) about 300 mg of at least one surfactant and (d) a pharmaceutically acceptable vehicle.

In one embodiment, the injectable composition of the present invention comprises (a) about 5 mg of metolazone, (b) about 1000 mg of solvent, (c) about 100 mg of surfactant and (d) a pharmaceutically acceptable vehicle. In an embodiment, the composition has a volume of about 1.6 ml.

In one more embodiment, the injectable composition of the present invention comprises (a) about 5 mg of metolazone, (b) about 800 mg of solvent, (c) about 300 mg of surfactant and (d) a pharmaceutically acceptable vehicle. In an embodiment, the composition has a volume of about 1.6 ml.

In a further embodiment, the injectable composition of the present invention comprises (a) about 5 mg of metolazone, (b) about 600 mg of solvent, (c) about 400 mg of surfactant and (d) a pharmaceutically acceptable vehicle. In an embodiment, the composition has a volume of about 2 ml. In one more embodiment, the injectable composition of the present invention comprises (a) about 0.01 mg to about 20 mg of metolazone, (b) about 100 mg to about 600 mg of at least one solvent comprising at least one of ethanol, propylene glycol, and polyethylene glycol, (c) about 100 mg to about 300 mg of polysorbate 80 and (d) water.

In another embodiment, the injectable composition of the present invention comprises (a) about 5 mg of metolazone, (b) about 100 mg to about 600 mg of at least one solvent comprising at least one of ethanol, propylene glycol, and polyethylene glycol, (c) about 100 mg to about 300 mg of polysorbate 80 and (d) water.

In one embodiment, the injectable composition of the present invention comprises (a) about 5 mg of metolazone, (b) about 200 mg of at least one solvent comprising at least one of ethanol, propylene glycol, and polyethylene glycol, (c) about 100 mg to about 300 mg of polysorbate 80 and (d) water.

In one embodiment, the injectable composition of the present invention comprises (a) about 5 mg of metolazone, (b) about 300 mg of at least one solvent comprising at least one of ethanol, propylene glycol, and polyethylene glycol, (c) about 100 mg to about 300 mg of polysorbate 80 and (d) water.

In one embodiment, the injectable composition of the present invention comprises (a) about 5 mg of metolazone, (b) about 400 mg of at least one solvent comprising at least one of ethanol, propylene glycol, and polyethylene glycol, (c) about 100 mg to about 300 mg of polysorbate 80 and (d) water.

In another embodiment, the injectable composition of the present invention comprises (a) about 5 mg of metolazone, (b) about 200 mg of polyethylene glycol, (c) about 100 mg of polysorbate 80 and (d) water.

In another embodiment, the injectable composition of the present invention comprises (a) about 5 mg of metolazone, (b) about 200 mg of polyethylene glycol, (c) about 200 mg of polysorbate 80 and (d) water.

In another embodiment, the injectable composition of the present invention comprises (a) about 5 mg of metolazone, (b) about 200 mg of polyethylene glycol, (c) about 300 mg of polysorbate 80 and (d) water.

In another embodiment, the injectable composition of the present invention comprises (a) about 5 mg of metolazone, (b) about 300 mg of polyethylene glycol, (c) about 100 mg of polysorbate 80 and (d) water.

In one embodiment, the injectable composition of the present invention comprises (a) about 5 mg of metolazone, (b) about 300 mg of polyethylene glycol, (c) about 200 mg of polysorbate 80 and (d) water.

In one more embodiment, the injectable composition of the present invention comprises (a) about 5 mg of metolazone, (b) about 300 mg of polyethylene glycol, (c) about 300 mg of polysorbate 80 and (d) water.

In one embodiment, the injectable composition of the present invention comprises (a) about 5 mg of metolazone, (b) about 400 mg of polyethylene glycol, (c) about 100 mg of polysorbate 80 and (d) water.

In one embodiment, the injectable composition of the present invention comprises (a) about 5 mg of metolazone, (b) about 400 mg of polyethylene glycol, (c) about 200 mg of polysorbate 80 and (d) water.

In one embodiment, the injectable composition of the present invention comprises (a) about 5 mg of metolazone, (b) about 400 mg of polyethylene glycol, (c) about 300 mg of polysorbate 80 and (d) water.

In another embodiment, the injectable composition of the present invention comprises (a) about 5 mg of metolazone, (b) about 200 mg of ethanol, (c) about 100 mg of polysorbate 80 and (d) water.

In an embodiment, the injectable composition of the present invention comprises (a) about 5 mg of metolazone, (b) about 200 mg of ethanol, (c) about 125 mg of polysorbate 80 and (d) water.

In another embodiment, the injectable composition of the present invention comprises (a) about 5 mg of metolazone, (b) about 200 mg of ethanol, (c) about 150 mg of polysorbate 80 and (d) water.

In another embodiment, the injectable composition of the present invention comprises (a) about 5 mg of metolazone, (b) about 200 mg of ethanol, (c) about 175 mg of polysorbate 80 and (d) water.

In another embodiment, the injectable composition of the present invention comprises (a) about 5 mg of metolazone, (b) about 200 mg of ethanol, (c) about 200 mg of polysorbate 80 and (d) water.

In another embodiment, the injectable composition of the present invention comprises (a) about 5 mg of metolazone, (b) about 200 mg of ethanol, (c) about 300 mg of polysorbate 80 and (d) water.

In another embodiment, the injectable composition of the present invention comprises (a) about 5 mg of metolazone, (b) about 300 mg of ethanol, (c) about 100 mg of polysorbate 80 and (d) water.

In one embodiment, the injectable composition of the present invention comprises (a) about 5 mg of metolazone, (b) about 300 mg of ethanol, (c) about 125 mg of polysorbate 80 and (d) water.

In another embodiment, the injectable composition of the present invention comprises (a) about 5 mg of metolazone, (b) about 300 mg of ethanol, (c) about 150 mg of polysorbate 80 and (d) water.

In a further embodiment, the injectable composition of the present invention comprises (a) about 5 mg of metolazone, (b) about 300 mg of ethanol, (c) about 175 mg of polysorbate 80 and (d) water.

In one embodiment, the injectable composition of the present invention comprises (a) about 5 mg of metolazone, (b) about 300 mg of ethanol, (c) about 200 mg of polysorbate 80 and (d) water.

In one more embodiment, the injectable composition of the present invention comprises (a) about 5 mg of metolazone, (b) about 300 mg of ethanol, (c) about 300 mg of polysorbate 80 and (d) water.

In one embodiment, the injectable composition of the present invention comprises (a) about 5 mg of metolazone, (b) about 400 mg of ethanol, (c) about 100 mg of polysorbate 80 and (d) water.

In one more embodiment, the injectable composition of the present invention comprises (a) about 5 mg of metolazone, (b) about 400 mg of ethanol, (c) about 125 mg of polysorbate 80 and (d) water.

In another embodiment, the injectable composition of the present invention comprises (a) about 5 mg of metolazone, (b) about 400 mg of ethanol, (c) about 150 mg of polysorbate 80 and (d) water.

In a further embodiment, the injectable composition of the present invention comprises (a) about 5 mg of metolazone, (b) about 400 mg of ethanol, (c) about 175 mg of polysorbate 80 and (d) water.

In one embodiment, the injectable composition of the present invention comprises (a) about 5 mg of metolazone, (b) about 400 mg ethanol, (c) about 200 mg of polysorbate 80 and (d) water.

In one embodiment, the injectable composition of the present invention comprises (a) about 5 mg of metolazone, (b) about 400 mg of ethanol, (c) about 300 mg of polysorbate 80 and (d) water.

In one another embodiment, the injectable composition of the present invention comprises (a) about 5 mg of metolazone, (b) about 300 mg of propylene glycol, (c) about 300 mg of polysorbate 80 and (d) water.

In one embodiment, the injectable composition of the present invention comprises (a) about 5 mg of metolazone, (b) about 1000 mg of ethanol, (c) about 100 mg of PEG 35 Castor oil and (d) water. In an embodiment, the composition has a volume of about 1.6 ml.

In one more embodiment, the injectable composition of the present invention comprises (a) about 5 mg of metolazone, (b) about 800 mg of ethanol, (c) about 300 mg of PEG 35 Castor oil and (d) water. In an embodiment, the composition has a volume of about 1.6 ml.

In a further embodiment, the injectable composition of the present invention comprises (a) about 5 mg of metolazone, (b) about 600 mg of ethanol, (c) about 400 mg of PEG 35 Castor oil and (d) water. In an embodiment, the composition has a volume of about 2 ml.

In one embodiment, the injectable composition of the present invention comprises (a) about 0.01 mg to about 20 mg of metolazone, (b) about 0.1% w/v to about 70% w/v of at least one solvent, (c) about 0.1% w/v to about 50% w/v of at least one surfactant and (d) a pharmaceutically acceptable vehicle.

In a further embodiment, the injectable composition of the present invention comprises (a) about 0.01 mg to about 20 mg of metolazone, (b) about 10% w/v to about 60% w/v of at least one solvent, (c) about 0.1% w/v to about 50% w/v of at least one surfactant and (d) a pharmaceutically acceptable vehicle.

In one more embodiment, the injectable composition of the present invention comprises (a) about 0.01 mg to about 20 mg of metolazone, (b) about 10% w/v to about 60% w/v of at least one solvent, (c) about 10% w/v of at least one surfactant and (d) a pharmaceutically acceptable vehicle.

In one embodiment, the injectable composition of the present invention comprises (a) about 0.01 mg to about 20 mg of metolazone, (b) about 10% w/v to about 60% w/v of at least one solvent, (c) about 12.5% w/v of at least one surfactant and (d) a pharmaceutically acceptable vehicle.

In one another embodiment, the injectable composition of the present invention comprises (a) about 0.01 mg to about 20 mg of metolazone, (b) about 10% w/v to about 60% w/v of at least one solvent, (c) about 15% w/v of at least one surfactant and (d) a pharmaceutically acceptable vehicle.

In one more embodiment, the injectable composition of the present invention comprises (a) about 0.01 mg to about 20 mg of metolazone, (b) about 10% w/v to about 60% w/v of at least one solvent, (c) about 17.5% w/v of at least one surfactant and (d) a pharmaceutically acceptable vehicle.

In another embodiment, the injectable composition of the present invention comprises (a) about 0.01 mg to about 20 mg of metolazone, (b) about 10% w/v to about 60% w/v of at least one solvent, (c) about 20% w/v of at least one surfactant and (d) a pharmaceutically acceptable vehicle.

In an embodiment, the injectable composition of the present invention comprises (a) about 0.01 mg to about 20 mg of metolazone, (b) about 10% w/v to about 60% w/v of at least one solvent, (c) about 30% w/v of at least one surfactant and (d) a pharmaceutically acceptable vehicle.

In a further embodiment, the injectable composition of the present invention comprises (a) about 5 mg of metolazone, (b) about 20% w/v of at least one solvent, (c) about 10% w/v of at least one surfactant and (d) a pharmaceutically acceptable vehicle.

In one more embodiment, the injectable composition of the present invention comprises (a) about 5 mg of metolazone, (b) about 20% w/v of at least one solvent, (c) about 12.5% w/v of at least one surfactant and (d) a pharmaceutically acceptable vehicle.

In one another embodiment, the injectable composition of the present invention comprises (a) about 5 mg of metolazone, (b) about 20% w/v of at least one solvent, (c) about 15% w/v of at least one surfactant and (d) a pharmaceutically acceptable vehicle.

In an embodiment, the injectable composition of the present invention comprises (a) about 5 mg of metolazone, (b) about 20% w/v of at least one solvent, (c) about 17.5% w/v of at least one surfactant and (d) a pharmaceutically acceptable vehicle.

In one embodiment, the injectable composition of the present invention comprises (a) about 5 mg of metolazone, (b) about 20% w/v of at least one solvent, (c) about 20% w/v of at least one surfactant and (d) a pharmaceutically acceptable vehicle.

In another embodiment, the injectable composition of the present invention comprises (a) about 5 mg of metolazone, (b) about 20% w/v of at least one solvent, (c) about 30% w/v of at least one surfactant and (d) a pharmaceutically acceptable vehicle.

In a further embodiment, the injectable composition of the present invention comprises (a) about 5 mg of metolazone, (b) about 30% w/v of at least one solvent, (c) about 10% w/v of at least one surfactant and (d) a pharmaceutically acceptable vehicle.

In an embodiment, the injectable composition of the present invention comprises (a) about 5 mg of metolazone, (b) about 30% w/v of at least one solvent, (c) about 12.5% w/v of at least one surfactant and (d) a pharmaceutically acceptable vehicle.

In a further embodiment, the injectable composition of the present invention comprises (a) about 5 mg of metolazone, (b) about 30% w/v of at least one solvent, (c) about 15% w/v of at least one surfactant and (d) a pharmaceutically acceptable vehicle.

In an embodiment, the injectable composition of the present invention comprises (a) about 5 mg of metolazone, (b) about 30% w/v of at least one solvent, (c) about 17.5% w/v of at least one surfactant and (d) a pharmaceutically acceptable vehicle.

In one embodiment, the injectable composition of the present invention comprises (a) about 5 mg of metolazone, (b) about 30% w/v of at least one solvent, (c) about 20% w/v of at least one surfactant and (d) a pharmaceutically acceptable vehicle.

In another embodiment, the injectable composition of the present invention comprises (a) about 5 mg of metolazone, (b) about 30% w/v of at least one solvent, (c) about 30% w/v of at least one surfactant and (d) a pharmaceutically acceptable vehicle.

In a further embodiment, the injectable composition of the present invention comprises (a) about 5 mg of metolazone, (b) about 40% w/v of at least one solvent, (c) about 10% w/v of at least one surfactant and (d) a pharmaceutically acceptable vehicle.

In an embodiment, the injectable composition of the present invention comprises (a) about 5 mg of metolazone, (b) about 40% w/v of at least one solvent, (c) about 12.5% w/v of at least one surfactant and (d) a pharmaceutically acceptable vehicle.

In a further embodiment, the injectable composition of the present invention comprises (a) about 5 mg of metolazone, (b) about 40% w/v of at least one solvent, (c) about 15% w/v of at least one surfactant and (d) a pharmaceutically acceptable vehicle.

In another embodiment, the injectable composition of the present invention comprises (a) about 5 mg of metolazone, (b) about 40% w/v of at least one solvent, (c) about 17.5% w/v of at least one surfactant and (d) a pharmaceutically acceptable vehicle.

In one embodiment, the injectable composition of the present invention comprises (a) about 5 mg of metolazone, (b) about 40% w/v of at least one solvent, (c) about 20% w/v of at least one surfactant and (d) a pharmaceutically acceptable vehicle.

In another embodiment, the injectable composition of the present invention comprises (a) about 5 mg of metolazone, (b) about 40% w/v of at least one solvent, (c) about 30% w/v of at least one surfactant and (d) a pharmaceutically acceptable vehicle.

In one embodiment, the injectable composition of the present invention comprises (a) about 5 mg of metolazone, (b) about 62.5% w/v of solvent, (c) about 6.25% w/v of surfactant and (d) a pharmaceutically acceptable vehicle.

In one more embodiment, the injectable composition of the present invention comprises (a) about 5 mg of metolazone, (b) about 50% w/v of solvent, (c) about 18.75% w/v of surfactant and (d) a pharmaceutically acceptable vehicle.

In a further embodiment, the injectable composition of the present invention comprises (a) about 5 mg of metolazone, (b) about 30% w/v of solvent, (c) about 20% w/v of surfactant and (d) a pharmaceutically acceptable vehicle.

In a further embodiment, the injectable composition of the present invention comprises (a) about 0.01 mg to about 20 mg of metolazone, (b) about 10% w/v to about 60% w/v of at least one solvent comprising at least one of ethanol, propylene glycol, and polyethylene glycol, (c) about 10% w/v to about 30% w/v of polysorbate 80 and (d) water.

In another embodiment, the injectable composition of the present invention comprises (a) about 5 mg of metolazone, (b) about 10% w/v to about 60% w/v of at least one solvent comprising at least one of ethanol, propylene glycol, and polyethylene glycol, (c) about 10% w/v to about 30% w/v of polysorbate 80 and (d) water.

In one embodiment, the injectable composition of the present invention comprises (a) about 5 mg of metolazone, (b) about 20% w/v of at least one solvent comprising at least one of ethanol, propylene glycol, and polyethylene glycol, (c) about 10% w/v to about 30% w/v of polysorbate 80 and (d) water.

In one more embodiment, the injectable composition of the present invention comprises (a) about 5 mg of metolazone, (b) about 30% w/v of at least one solvent comprising at least one of ethanol, propylene glycol, and polyethylene glycol, (c) about 10% w/v to about 30% w/v of polysorbate 80 and (d) water.

In one embodiment, the injectable composition of the present invention comprises (a) about 5 mg of metolazone, (b) about 40% w/v of at least one solvent comprising at least one of ethanol, propylene glycol, and polyethylene glycol, (c) about 10% w/v to about 30% w/v of polysorbate 80 and (d) water.

In a further embodiment, the injectable composition of the present invention comprises (a) about 5 mg of metolazone, (b) about 20% w/v of polyethylene glycol, (c) about 10% w/v of polysorbate 80 and (d) water.

In one embodiment, the injectable composition of the present invention comprises (a) about 5 mg of metolazone, (b) about 20% w/v of polyethylene glycol, (c) about 20% w/v of polysorbate 80 and (d) water.

In an embodiment, the injectable composition of the present invention comprises (a) about 5 mg of metolazone, (b) about 20% w/v of polyethylene glycol, (c) about 30% w/v of polysorbate 80 and (d) water.

In a further embodiment, the injectable composition of the present invention comprises (a) about 5 mg of metolazone, (b) about 30% w/v of polyethylene glycol, (c) about 10% w/v of polysorbate 80 and (d) water.

In another embodiment, the injectable composition of the present invention comprises (a) about 5 mg of metolazone, (b) about 30% w/v of polyethylene glycol, (c) about 20% w/v of polysorbate 80 and (d) water.

In one embodiment, the injectable composition of the present invention comprises (a) about 5 mg of metolazone, (b) about 30% w/v of polyethylene glycol, (c) about 30% w/v of polysorbate 80 and (d) water.

In a further embodiment, the injectable composition of the present invention comprises (a) about 5 mg of metolazone, (b) about 40% w/v of polyethylene glycol, (c) about 10% w/v of polysorbate 80 and (d) water.

In another embodiment, the injectable composition of the present invention comprises (a) about 5 mg of metolazone, (b) about 40% w/v of polyethylene glycol, (c) about 20% w/v of polysorbate 80 and (d) water.

In one embodiment, the injectable composition of the present invention comprises (a) about 5 mg of metolazone, (b) about 40% w/v of polyethylene glycol, (c) about 30% w/v of polysorbate 80 and (d) water.

In a further embodiment, the injectable composition of the present invention comprises (a) about 5 mg of metolazone, (b) about 20% w/v of ethanol, (c) about 10% w/v of polysorbate 80 and (d) water.

In one embodiment, the injectable composition of the present invention comprises (a) about 5 mg of metolazone, (b) about 20% w/v of ethanol, (c) about 12.5% w/v of polysorbate 80 and (d) water.

In an embodiment, the injectable composition of the present invention comprises (a) about 5 mg of metolazone, (b) about 20% w/v of ethanol, (c) about 15% w/v of polysorbate 80 and (d) water.

In a further embodiment, the injectable composition of the present invention comprises (a) about 5 mg of metolazone, (b) about 20% w/v of ethanol, (c) about 17.5% w/v of polysorbate 80 and (d) water.

In one embodiment, the injectable composition of the present invention comprises (a) about 5 mg of metolazone, (b) about 20% w/v of ethanol, (c) about 20% w/v of polysorbate 80 and (d) water.

In an embodiment, the injectable composition of the present invention comprises (a) about 5 mg of metolazone, (b) about 20% w/v of ethanol, (c) about 30% w/v of polysorbate 80 and (d) water.

In a further embodiment, the injectable composition of the present invention comprises (a) about 5 mg of metolazone, (b) about 30% w/v of ethanol, (c) about 10% w/v of polysorbate 80 and (d) water.

In an embodiment, the injectable composition of the present invention comprises (a) about 5 mg of metolazone, (b) about 30% w/v of ethanol, (c) about 12.5% w/v of polysorbate 80 and (d) water.

In one embodiment, the injectable composition of the present invention comprises (a) about 5 mg of metolazone, (b) about 30% w/v of ethanol, (c) about 15% w/v of polysorbate 80 and (d) water.

In one more embodiment, the injectable composition of the present invention comprises (a) about 5 mg of metolazone, (b) about 30% w/v of ethanol, (c) about 17.5% w/v of polysorbate 80 and (d) water.

In another embodiment, the injectable composition of the present invention comprises (a) about 5 mg of metolazone, (b) about 30% w/v of ethanol, (c) about 20% w/v of polysorbate 80 and (d) water.

In one embodiment, the injectable composition of the present invention comprises (a) about 5 mg of metolazone, (b) about 30% w/v of ethanol, (c) about 30% w/v of polysorbate 80 and (d) water.

In a further embodiment, the injectable composition of the present invention comprises (a) about 5 mg of metolazone, (b) about 40% w/v of ethanol, (c) about 10% w/v of polysorbate 80 and (d) water.

In one embodiment, the injectable composition of the present invention comprises (a) about 5 mg of metolazone, (b) about 40% w/v of ethanol, (c) about 12.5% w/v of polysorbate 80 and (d) water.

In an embodiment, the injectable composition of the present invention comprises (a) about 5 mg of metolazone, (b) about 40% w/v of ethanol, (c) about 15% w/v of polysorbate 80 and (d) water.

In one another embodiment, the injectable composition of the present invention comprises (a) about 5 mg of metolazone, (b) about 40% w/v of ethanol, (c) about 17.5% w/v of polysorbate 80 and (d) water.

In another embodiment, the injectable composition of the present invention comprises (a) about 5 mg of metolazone, (b) about 40% w/v of ethanol, (c) about 20% w/v of polysorbate 80 and (d) water.

In one embodiment, the injectable composition of the present invention comprises (a) about 5 mg of metolazone, (b) about 40% w/v of ethanol, (c) about 30% w/v of polysorbate 80 and (d) water.

In one embodiment, the injectable composition of the present invention comprises (a) about 5 mg of metolazone, (b) about 30% w/v of propylene glycol, (c) about 30% w/v of polysorbate 80 and (d) water.

In one embodiment, the injectable composition of the present invention comprises (a) about 0.1 mg to about 10 mg of metolazone, (b) about 0.1% w/v to about 70% w/v of at least one solvent, (c) about 0.1% w/v to about 50% w/v of at least one surfactant and (d) a pharmaceutically acceptable vehicle.

In another embodiment, the injectable composition of the present invention comprises (a) about 0.1 mg to about 10 mg of metolazone, (b) about 30% w/v to about 65% w/v of at least one solvent, (c) about 5% w/v to about 30% w/v of at least one surfactant and (d) a pharmaceutically acceptable vehicle.

In one more embodiment, the injectable composition of the present invention comprises (a) about 0.1 mg to about 10 mg of metolazone, (b) about 0.1% w/v to about 70% w/v of ethanol, (c) about 0.1% w/v to about 50% w/v of PEG 35 castor oil and (d) a pharmaceutically acceptable vehicle.

In a further embodiment, the injectable composition of the present invention comprises (a) about 0.1 mg to about 10 mg of metolazone, (b) about 30% w/v to about 65% w/v of ethanol, (c) about 5% w/v to about 30% w/v of PEG 35 castor oil and (d) a pharmaceutically acceptable vehicle.

In an embodiment, the injectable composition of the present invention comprises (a) about 0.1 mg to about 10 mg of metolazone, (b) about 62.5% w/v of ethanol, (c) about 6.25% w/v of PEG 35 castor oil and (d) water.

In one more embodiment, the injectable composition of the present invention comprises (a) about 0.1 mg to about 10 mg of metolazone, (b) about 50% w/v of ethanol, (c) about 18.75% w/v of PEG 35 castor oil and (d) water.

In a further embodiment, the injectable composition of the present invention comprises (a) about 0.1 mg to about 10 mg of metolazone, (b) about 30% w/v of ethanol, (c) about 20% w/v of PEG 35 castor oil and (d) water.

In one embodiment, the injectable composition of the present invention comprises (a) about 5 mg of metolazone, (b) about 62.5% w/v of ethanol, (c) about 6.25% w/v of PEG 35 Castor oil and (d) water.

In one more embodiment, the injectable composition of the present invention comprises (a) about 5 mg of metolazone, (b) about 50% w/v of ethanol, (c) about 18.75% w/v of PEG 35 Castor oil and (d) water.

In a further embodiment, the injectable composition of the present invention comprises (a) about 5 mg of metolazone, (b) about 30% w/v of ethanol, (c) about 20% w/v of PEG 35 Castor oil and (d) water.

In one more embodiment, the injectable composition of the present invention is stable for at least 1 hour after dilution with a compatible admixture diluent.

In an embodiment, the injectable composition of the present invention is aqueous.

The injectable composition of the present invention may be diluted with compatible admixture diluents well known in the art before administering to a subject in need thereof.

The pH of the injectable composition of the present invention may be between about 6 to about 8.

The injectable composition of the present invention may demonstrate an osmolality between about 300 to about 350 osmole/kg upon dilution with compatible admixture diluents.

The injectable composition comprising metolazone remains stable for at least 1 hour after dilution with compatible admixture diluent. The inventors have found that the combination of surfactant and solvent is critical for maintaining the stability of the injectable composition upon dilution with the compatible admixture diluent.

The term “stable” refers to an injection composition of present invention which is physically as well as chemically stable as demonstrated by compliance to acceptable specification when the composition is stored at convenient temperature, such as between about 0 °C and about 60 °C, for a commercially reasonable period of time, such as at least about 1 day, at least about 1 week, at least about 1 month, at least about 3 months, at least about 6 months, at least about 1 year, or at least about 2 years. The term “stable” also refers to an injectable composition as per the present invention which exhibits physical stability for a sufficiently long time to allow for infusion of the product upon dilution with compatible admixture diluent such as sterile water for injection, saline or dextrose. The injectable compositions of the present invention remain stable after dilution with a compatible admixture diluent for a sufficient time to allow for infusion to be completed. Suitably, the injectable composition of metolazone of present invention remains physically stable, with no precipitation or crystallization or color change upon storage and the shelf life period of 18-24 months when stored at 2-8 °C. Suitably, the injectable composition of metolazone remains chemically stable when stored at 2-8 °C, wherein various parameters such as the drug content (assay of metolazone) and content of related substances, i.e. known and unknown impurities remains within specified limits such as those specified according to ICH guidelines, upon storage for prolonged period of time such as for at least 12 months, preferably for 18 months, more preferably 24 months or longer. The injectable composition of the present invention remains stable for at least 1 hour after dilution with a compatible admixture diluent.

The injectable composition of present invention is substantially free of impurities. For purposes of the present invention, “substantially free of impurities’ shall be understood to include metolazone containing injectable compositions in which the amount of total impurities is less than about 5% of the sum of peak areas of all degradants, as calculated on a normalized peak area response (“PAR) basis as determined by high performance liquid chromatograph (“HPLC) after a period of about 18 months at a temperature of from about 2 °C to about 8 °C. The amount of impurities is further calculated as being based upon the original amount of metolazone (or salt thereof) being present in the composition. Preferably, the said injectable compositions of metolazone prevent degradation of metolazone such that not more than 2%, not more than 1%, not more than 0.4%, not more than 0.2% of metolazone impurity or impurities are formed over the storage period. In yet another preferred embodiment the value of assay of metolazone remains within the specified limit of 90-110% by weight of the label claim.

The ICH storage stability studies were performed on the injectable composition of the present invention packaged in the proposed commercial primary packaging and closure system. The stability study samples were stored at 2-8 °C, 25 °C, 30 °C, and 40 °C. The necessary parameters viz., assay, related substances, particle size distribution, pH, and osmolality were tested and found to be within specification at both stability conditions. The stability data suggested long term stability at 2-8 °C. In some preferred aspects of the invention, the time for which long term storage are contemplated include periods of at least about 24 months or longer with such that the composition is substantially free of impurities when stored at 2-8 °C.

While not wishing to be bound by any theory whatsoever, it is believed that the use of excipients such as surfactants and solvents in preparing injectable composition comprising metolazone play a significant role in reducing the degradation of metolazone thereby prolonging the shelf-life of said metolazone compositions. It is also believed that the combination of surfactant and solvent is critical for the physical stability of the injectable compositions of the present invention when diluted with a compatible admixture diluent for infusion to a subject in need thereof.

The addition of the components for the preparation of stable injectable composition can be achieved by methods known in the art.

In an embodiment, the injectable composition of the present invention can be prepared by a process comprising (a) preparing a mixture of at least one solvent and at least one surfactant; (b) dissolving metolazone in solution of step (a); (c) making up volume of solution of step (b) with water; (d) sterilizing the solution of step (c) followed by filling in appropriate container.

In a specific embodiment, the injectable composition of the present invention can be prepared by a process comprising (a) preparing a mixture of at least one solvent comprising at least one of ethanol, polyethylene glycol, and propylene glycol and at least one surfactant comprising polysorbate 80; (b) dissolving metolazone in solution of step (a); (c) making up volume of solution of step (b) with water; (d) sterilizing the solution of step (c) followed by filling in appropriate container. Preferably, the process of preparation of the composition of the present invention is carried out under nitrogen purging or blanketing.

The injectable compositions of the present invention can be packaged in any suitable sterile vial or prefilled syringe or container fit for the sterile storage of pharmaceuticals. The stable injectable composition of the present invention can be provided in a kit or package that includes a container enclosing the composition. Suitable containers can be glass vials, i.e., Schott treated vials, molded glass vials, CZ resin vials, polypropylene or polyethylene vials or other special purpose containers. Suitable containers can be prefilled syringes such as glass prefilled syringes, plastic prefilled syringes. Containers are of a size sufficient to hold one or more doses of metolazone. The container may be part of a syringe or separate from the syringe. The kit or package also includes a needle that can be suitably mounted to the syringe. The size of the needle, in some embodiments, is equal to or smaller than 18G, 19G, 20G, 21G, 22G, 23G, 24G, or 25G. In one embodiment, the needle has a size that is 20G or smaller. In one embodiment, the needle has a size that is 21G or smaller. In one embodiment, the needle has a size that is 22G or smaller. In one embodiment, the needle has a size that is 23G or smaller. In one embodiment, the injectable composition of the present invention may be administered without the need of an in-line filter during administration, demonstrating the absence of particulate matter throughout its shelf life. In another embodiment, the injectable composition of the present invention may be filtered by using an in-line filter before administration.

The present invention further provides methods of treating a patient suffering from salt and water retention by injection of the composition of the present invention.

The term “treatment of patient suffering from salt and water retention” includes, but not limited to treatment of edema accompanying congestive heart failure; and edema accompanying renal diseases, including the nephrotic syndrome and states of diminished renal function.

### EXAMPLES

The following examples are for the purpose of illustration of the invention only and are not intended to limit the scope of the present invention in any manner whatsoever.

### Example 1

The below table represents injectable composition using only solvents and without addition of any surfactant.

| Ingredients | A | B | C |
|---|---|---|---|
| Metolazone | 5 mg | 5 mg | 5 mg |
| Propylene Glycol | 500 mg | — | — |
| PEG 400 | — | — | 500 mg |
| Ethanol | — | 500 mg | — |
| Water | q.s 1 ml | q.s 1 ml | q.s 1 ml |
| Observation | Clear solution | Clear solution | Clear solution |
| At room | Precipitated | Precipitated | Precipitated |
| temperature (13 | at 24 hr | | |
| day) | | | |
| pH | 5.52 | 6.28 | 6.28 |
| Upon dilution with | — | Precipitated | Precipitated |
| compatible | | | |
| admixture diluents | | | |
| (10 day) | | | |

The above table demonstrates that compositions prepared with only solvent (without any surfactants) did not show adequate stability and precipitated within 13 days and also did not exhibit sufficient stability upon dilution with compatible admixture diluents.

### Example 2

The below table represents injectable composition with only surfactants and without any solvents

| Ingredients | A | B | C | D | E |
|---|---|---|---|---|---|
| Metolazone | 5 mg | 5 mg | 5 mg | 5 mg | 5 mg |
| Polysorbate | 500 mg | 400 mg | 300 mg | 200 mg | 100 mg |
| 80 | | | | | |
| Water | q.s 1 ml | q.s 1 ml | q.s 1 ml | q.s 1 ml | q.s 1 ml |
| Observation | Clear Gel | Clear | Precipi- | Metola- | Metola- |
| | like | viscous | tated after | zone not | zone not |
| | consistency | solution | 30 min | soluble | soluble |
| At room | Clear Gel | Clear | — | — | — |
| temperature | like | viscous | | | |
| (7 day) | consistency | solution | | | |
| At 2-8 °C | Clear Gel | Clear | — | — | — |
| (7 day) | like | viscous | | | |
| | consistency | solution | | | |
| pH | 6.68 | 6.25 | 5.99 | — | — |

Upon dilution with compatible admixture diluents

| | | | | | |
|---|---|---|---|---|---|
| 10-day data | Clear | Clear | — | — | — |
| | solution | solution | | | |

The above table demonstrates that the composition with Polysorbate 80 in amounts greater than 400 mg/ml were found to be stable and precipitation of metolazone was not observed even after dilution with compatible admixture diluents. However, these high quantities of Polysorbate 80 resulted in very viscous compositions, rendering them difficult to administer.

### Example 3

The below table represents injectable composition comprising a combination of polysorbate 80 in varying concentrations with fixed concentrations of ethanol

| Ingredients | A | | B | | C | | D | | E | | F | | G | | H | |
|---|---|---|---|---|---|---|---|---|---|---|---|---|---|---|---|---|
| Metolazone | 5 | mg | 5 | mg | 5 | mg | 5 | mg | 5 | mg | 5 | mg | 5 | mg | 5 | mg |
| Ethanol | 300 | mg | 300 | mg | 300 | mg | 300 | mg | 300 | mg | 300 | mg | 400 | mg | 500 | mg |
| Polysorbate 80 | 300 | mg | 200 | mg | 175 | mg | 150 | mg | 125 | mg | 100 | mg | 100 | mg | — | |
| Water | q.s 1 | ml | q.s 1 | ml | q.s 1 | ml | q.s 1 | ml | q.s 1 | ml | q.s 1 | ml | q.s 1 | ml | q.s 1 | ml |

| Observation | Clear | Clear | Clear | Clear | Clear | | Clear | | Clear | | Clear | |
|---|---|---|---|---|---|---|---|---|---|---|---|---|
| | solution | solution | solution | solution | solution | | solution | | solution | | solution | |
| At room | Clear | Clear | Clear | Clear | Precip- | | Precip- | | Precip- | | Precip- | |
| temperature | solution | solution | solution | solution | itated | | itated | | itated | | itated | |
| | | | | | after | | after | | after | | after | |
| | | | | | 7 | days | 7 | days | 1 | month | 24 | hours |

| At 2-8 °C | Clear | Clear | Clear | Clear | Precip- | | Precip- | | Precip- | | — |
|---|---|---|---|---|---|---|---|---|---|---|---|
| | solution | solution | solution | solution | itated | | itated | | itated | | |
| | | | | | after | | after | | after | | |
| | | | | | 7 | days | 7 | days | 1 | month | |

| | | | | | | | | |
|---|---|---|---|---|---|---|---|---|
| pH | 7.10 | 6.77 | 6.41 | 6.29 | 6.14 | 6.55 | 6.68 | 6.28 |
| Upon | Clear | Clear | Precip- | Precip- | Precip- | Precip- | — | — |
| dilution with | solution | solution | itated | itated | itated | itated | | |
| compatible | | | | | | | | |
| admixture | | | | | | | | |
| diluents | | | | | | | | |
| (7 days) | | | | | | | | |

### Manufacturing Process:

(a) A mixture of ethanol and polysorbate 80 was prepared.
(b) Metolazone was dissolved in the solution of step (a)
(c) Volume was made up with water for injection and the resultant solution was sterilized and filled in appropriate containers.

The above table again confirms that injectable composition without a surfactant precipitated within 24 hours, and that an injectable composition with a solvent as well as a surfactant demonstrated good physical stability wherein precipitation was not observed even after dilution with compatible admixture diluents

### Example 4

The below table represents injectable composition comprising a combination of ethanol in varying concentrations with fixed concentrations of polysorbate 80

| Ingredients | A | B | C |
|---|---|---|---|
| Metolazone | 5 mg | 5 mg | 5 mg |
| Ethanol | 300 mg | 200 mg | 100 mg |
| Polysorbate 80 | 300 mg | 300 mg | 300 mg |
| Water | q.s 1 ml | q.s 1 ml | q.s 1 ml |
| Observation | Clear solution | Clear solution | Clear solution |
| At room temperature | Clear solution | Clear solution | Clear solution |
| (17 day) | | | |
| At 2-8 °C (17 day) | Clear solution | Clear solution | Clear solution |
| pH | 7.10 | 6.72 | 6.5 |
| Upon with | Clear solution | Clear solution | Clear solution |
| compatible admixture | | | |
| diluents | | | |
| (17 days) | | | |

### Manufacturing Process:

(a) A mixture of ethanol and polysorbate 80 was prepared.
(b) Metolazone was dissolved in the solution of step (a)
(c) Volume was made up with water for injection and the resultant solution was sterilized and filled in appropriate containers.

The above table demonstrates that the concentration of ethanol did not impact the physical stability of the injectable compositions with 300 mg/ml Polysorbate 80 and demonstrated good physical stability wherein precipitation was not observed until 17 days after dilution with compatible admixture diluents.

### Example 5

The below table represents injectable composition with combination of propylene glycol and Polysorbate 80

| Ingredients | A | | B | | C | | D | | E | | F | |
|---|---|---|---|---|---|---|---|---|---|---|---|---|
| Metolazone | 5 | mg | 5 | mg | 5 | mg | 5 | mg | 5 | mg | 5 | mg |
| Propylene | 300 | mg | 200 | mg | 100 | mg | 300 | mg | 300 | mg | 500 | mg |
| Glycol | | | | | | | | | | | | |
| Polysorbate | 300 | mg | 300 | mg | 300 | mg | 200 | mg | 100 | mg | — | |
| 80 | | | | | | | | | | | | |
| Water | q.s 1 | ml | q.s 1 | ml | q.s 1 | ml | q.s 1 | ml | q.s 1 | ml | q.s 1 | ml |

| | | | | | | |
|---|---|---|---|---|---|---|
| Observation | Clear | Clear | Clear | Clear | Clear | Precip- |
| | solution | solution | solution | solution | solution | itated |
| | | | | | | after 2 Hrs. |
| At room | Clear | Precip- | Precip- | Precip- | Precip- | Precip- |
| temperature | solution | itated | itated | itated | itated | itated |
| (17 day) | | | | | | |
| At 2-8 °C | Clear | — | — | — | — | — |
| (17 day) | solution | | | | | |
| pH | 6.88 | — | — | — | — | 5.52 |
| Upon | Clear | — | — | — | — | — |
| dilution with | solution | | | | | |
| compatible | | | | | | |
| admixture | | | | | | |
| diluents | | | | | | |
| (10 days) | | | | | | |

### Manufacturing Process:

(a) A mixture of propylene glycol and polysorbate 80 was prepared.
(b) Metolazone was dissolved in the solution of step (a)
(c) Volume was made up with water for injection and the resultant solution was sterilized and filled in appropriate containers.

The above table again confirms that injectable composition without a surfactant precipitated after 2 hours. It was observed that injectable composition containing 300 mg/ml polysorbate 80 and 300 mg/ml propylene glycol demonstrated good physical stability and precipitation was not observed until 10 days after dilution with compatible admixture diluents

### Example 6

The below table represents injectable composition with combination of polyethylene glycol and polysorbate 80

| Ingredients | A | | B | | C | | D | | E | | F | | G | | H | | I | |
|---|---|---|---|---|---|---|---|---|---|---|---|---|---|---|---|---|---|---|
| Metolazone | 5 | mg | 5 | mg | 5 | mg | 5 | mg | 5 | mg | 5 | mg | 5 | mg | 5 | mg | 5 | mg |
| Polyethylene | 400 | mg | 300 | mg | 200 | mg | 200 | mg | 100 | mg | 300 | mg | 300 | mg | 400 | mg | 500 | mg |
| glycol | | | | | | | | | | | | | | | | | | |
| (PEG 400) | | | | | | | | | | | | | | | | | | |
| Polysorbate | 400 | mg | 300 | mg | 200 | mg | 300 | mg | 300 | mg | 200 | mg | 100 | mg | 100 | mg | — | |
| 80 | | | | | | | | | | | | | | | | | | |
| Water | q.s 1 | ml | q.s 1 | ml | q.s 1 | ml | q.s 1 | ml | q.s 1 | ml | q.s 1 | ml | q.s 1 | ml | q.s 1 | ml | q.s 1 | ml |

| Observation | Clear | solution | Precip- | Clear | Clear | Clear | Clear | Clear | Clear | |
|---|---|---|---|---|---|---|---|---|---|---|
| | solution | Clear | itated | solution | solution | solution | solution | solution | solution | |
| At room | Clear | solution | Precip- | Clear | Precip- | Clear | Precip- | Clear | Precip- | |
| temperature | solution | Clear | itated | solution | itated | solution | itated | solution | itated | |
| (17 day) | | | | | | | | | | |
| At 2-8 °C | Clear | solution | Precip- | Clear | — | Clear | Precip- | Clear | Precip- | |
| (17 day) | solution | Clear | itated | solution | | solution | itated | solution | itated | |
| pH | 8.56 | 7.70 | 6.84 | 6.78 | 6.43 | 6.88 | 6.77 | 7.17 | 6.28 | |
| Upon | Clear | Clear | — | Clear | — | Clear | Precip- | — | Precip- | |
| dilution with | solution | solution | | solution | | solution | itated | | itated | |
| compatible | | | | | | | | | in | |
| admixture | | | | | | | | | 60 | min |

diluents
with
compatible
admixture
diluents
(17 days)

### Manufacturing Process:

(a) A mixture of polyethylene glycol (PEG 400) and polysorbate 80 was prepared.
(b) Metolazone was dissolved in the solution of step (a)
(c) Volume was made up with water for injection and the resultant solution was sterilized and filled in appropriate containers.

The above table confirms that an injectable composition without a surfactant precipitated. The data demonstrates that compositions with polyethylene glycol and in concentration of 200 mg/ml to 400 mg/ml with either 100 mg/ml, 200 mg/ml, or 300 mg/ml of polysorbate 80 demonstrated good physical stability and precipitation was not observed until 17 days after dilution with compatible admixture diluents.

### Example 7

The below table demonstrates stability data for Examples 6H and Example 3B:

Example Condition

| | | | | | Des- | | | Dide- | | | |
|---|---|---|---|---|---|---|---|---|---|---|---|
| | | | | | methyl | Para | Meta | hydro | | Single | |
| | | | | | metola- | metola- | metola- | metola- | Impurity | Maxi- | |
| | | Time | | Assay | zone | zone | zone | zone | at RRT | mum | Total |
| Example | Condition | point | pH | (%) | impurity | impurity | impurity | impurity | 0.78 | impurity | Impurity |
| Example | Initial | | 7.25 | 97.5 | ND | ND | 0.006 | 0.29 | ND | 0.15 | 0.44 |
| 6H | | | | | | | | | | (RRT 1.05) | |
| | 25 °C/ | 1M | 7.35 | 97.7 | ND | ND | ND | 0.249 | <0.1 | <0.1 (0.081) | 0.249 |
| | 60% RH | | | | | | | | (0.013) | (RRT 1.05) | |
| | 30 °C/ | 1M | 7.23 | 94.5 | ND | ND | ND | 0.261 | <0.1 | <0.1 (0.079) | 0.261 |
| | 75% RH | | | | | | | | (0.026) | (RRT 1.05) | |
| | 40 °C/ | 1M | 6.86 | 93.7 | ND | ND | ND | 0.365 | 0.134 | <0.1(0.075) | 0.499 |
| | 75% RH | | | | | | | | | (RRT 1.05) | |
| Example | Initial | | 6.65 | 100.9 | ND | 0.002 | 0.005 | 0.27 | ND | 0.141 | 0.41 |
| 3B | | | | | | | | | | (RRT 1.05) | |
| | 25 C/ | 1M | 6.53 | 102.2 | ND | ND | ND | 0.283 | <0.1 | <0.1 (0.087) | 0.283 |
| | 60% RH | | | | | | | | (0.01) | (RRT 1.055) | |
| | 30 °C/ | 1M | 6.56 | 100.8 | ND | ND | ND | 0.315 | <0.1 | <0.1 (0.096) | 0.315 |
| | 75% RH | | | | | | | | (0.018) | RRT 1.055) | |
| | 40 °C/ | 1M | 6.58 | 101.4 | ND | ND | ND | 0.406 | 0.102 | <0.1 (0.087) | 0.508 |
| | 75% RH | | | | | | | | | (RRT 1.055) | |

### Example 8

The below table represents injectable composition with combination of Cremophor EL (PEG 35 castor oil) and ethanol

| | A | | B | | C | |
|---|---|---|---|---|---|---|
| Ingredients | mg | % w/v | mg | % w/v | mg | % w/v |
| Metolazone | 5 | 0.31 | 5 | 0.31 | 5 | 0.25 |
| Ethanol | 1000 | 62.5 | 800 | 50 | 600 | 30 |
| Cremophor | 100 | 6.25 | 300 | 18.75 | 400 | 20 |
| EL (PEG 35 | | | | | | |
| Castor oil) | | | | | | |
| Water | q.s 1.6 ml | q.s 100 | q.s 1.6 ml | q.s 100 | q.s 2 ml | q.s 100 |

### Manufacturing Process:

(a) A mixture of ethanol and Cremophor EL (PEG 35 Castor oil) was prepared.
(b) Metolazone was dissolved in the solution of step (a).
(c) Volume was made up with water for injection and the resultant solution was sterilized and filled in appropriate containers.

The below table demonstrates stability data for Example 8A

| | | | | Des- | | | Dide- | Imp | | |
|---|---|---|---|---|---|---|---|---|---|---|
| | | | | methyl | Para | Meta | hydro | at | | |
| | | | Assay | Metola- | metol- | Metol- | Metola- | RRT | | Total |
| Stage | Condition | pH | % | zone | azone | azone | zone | 0.78 | SMI | Imp. |
| Initial | Initial | 7.85 | 102.2 | 0.008 | ND | 0.006 | 0.241 | ND | 0.166 | 0.52 |
| 2-8 °C | 1M | 7.81 | 103.2 | ND | ND | 0.007 | 0.256 | 0.007 | 0.082 | 0.26 |
| | 3M | 7.65 | 101.1 | ND | ND | 0.006 | 0.269 | 0.006 | 0.078 | 0.28 |
| | 6M | 7.44 | 102.0 | ND | ND | ND | 0.27 | ND | 0.07 | 0.27 |
| 25 C/ | 1M | 7.50 | 102.2 | ND | ND | 0.007 | 0.272 | 0.01 | 0.084 | 0.27 |
| 60% RH | 3M | 7.70 | 101.6 | 0.015 | ND | 0.007 | 0.322 | ND | 0.073 | 0.32 |
| | 6M | NP | 103.8 | ND | ND | ND | 0.36 | ND | 0.07 | 0.36 |
| 40 °C/ | 15D | 7.89 | 102.9 | 0.055 | ND | 0.007 | 0.357 | 0.029 | 0.081 | 0.36 |
| 75% RH | 1M | 7.87 | 101.7 | 0.056 | ND | 0.007 | 0.369 | 0.046 | 0.082 | 0.37 |

Although the invention herein has been described with reference to particular embodiments, it is to be understood that these embodiments are merely illustrative of the principles and application of the present invention. It is therefore to be understood that numerous modifications may be made to the illustrative embodiments and that other arrangements may be devised without departing from the spirit and scope of the present invention as described.

We claim:

1. A ready-to-dilute injectable composition comprising: (a) metolazone, (b) ethanol, (c) PEG 35 castor oil, and (d) water for injection.

2. A ready-to-dilute injectable composition comprising:
(a) metolazone in 0.27-0.35% w/v;
(b) ethanol in 30-65% w/v;
(c) PEG 35 castor oil in 1.0-30% w/v; and
(d) water for injection q.s.

3. The ready-to-dilute injectable composition of claim 2 formulated as a unit dosage form comprising a fill volume of 1.1-1.6 mL.

4. The composition of claim 2, formulated as a unit dosage form comprising metolazone in 0.1-10 mg.

5. The composition of claim 2, comprising ethanol in 60-65% w/v.

6. The composition of claim 2, comprising ethanol in 62.5% w/v.

7. The composition of claim 2, comprising PEG 35 castor oil in 6.25% w/v.

8. The composition of claim 2, formulated as a unit dosage form comprising: (a) 0.1-10 mg of metolazone, (b) 30-65% w/v of ethanol, (c) 5-30% w/v of PEG 35 castor oil, and (d) water for injection.

9. The injectable composition according to claim 2, wherein the composition is stable for at least 1 hour after dilution with a compatible admixture diluent.

10. The injectable composition according to claim 2, wherein the composition is aqueous.

11. The injectable composition according to claim 2, wherein after dilution with a compatible admixture diluent selected from 5% dextrose injection, 0.9% sodium chloride injection, or lactated Ringer's injection, the diluted composition remains clear and free from precipitation for at least 17 days at 25 °C and at 2-8 °C.

12. The injectable composition according to claim 2, comprising the ethanol and PEG 35 castor oil, in a weight ratio of 62.5:1.25 (ethanol:PEG castor oil).

13. The injectable composition according to claim 2, wherein the composition exhibits stability for at least 17 days at both 2-8 °C and 25 °C without visible precipitation, achieving a ready-to-dilute unit dosage form comprising metolazone in a concentration of 0.1-10 mg/mL.

14. A ready-to-dilute injectable composition comprising:
(a) metolazone in 0.27-0.35% w/v;
(b) ethanol in 60-65% w/v;
(c) PEG 35 castor oil in 1.0-1.5% w/v; and
(d) water for injection q.s.

15. The ready-to-dilute injectable composition of claim 14, formulated as a unit dosage form comprising a fill volume of 1.1-1.6 mL.

16. The injectable composition according to claim 14, formulated as a unit dosage form comprising 5 mg of metolazone.

17. The injectable composition according to claim 14, formulated as a unit dosage form comprising 4 mg of metolazone.

18. The injectable composition according to claim 14, formulated as a unit dosage form comprising metolazone in 0.1-10 mg.

19. The injectable composition according to claim 14, comprising ethanol in 30-65% w/v.

20. The injectable composition according to claim 14, comprising PEG 35 castor oil in 5-30% w/v.

21. The injectable composition according to claim 14, comprising ethanol in 62.5% w/v.

22. The injectable composition according to claim 14, comprising PEG 35 castor oil in 1.25% w/v.

23. The injectable composition according to claim 14, formulated as a unit dosage form comprising: (a) 0.1-10 mg of metolazone, (b) 30-65% w/v of ethanol, (c) 5-30% w/v of PEG 35 castor oil, and (d) water for injection.

24. The injectable composition according to claim 14, wherein the composition is stable for at least 1 hour after dilution with a compatible admixture diluent.

25. The injectable composition according to claim 14, wherein the composition is aqueous.

26. The injectable composition according to claim 14, wherein after dilution with a compatible admixture diluent selected from 5% dextrose injection, 0.9% sodium chloride injection, or lactated Ringer's injection, the diluted composition remains clear and free from precipitation for at least 17 days at 25 °C and at 2-8 °C.

27. The injectable composition according to claim 14, comprising ethanol and PEG 35 castor oil, in a weight ratio of 62.5:1.25 (ethanol:PEG 35 castor oil).

28. The injectable composition according to claim 14, wherein the composition exhibits stability for at least 17 days at both 2-8 °C and 25 °C without visible precipitation.

29. The injectable composition according to claim 14, wherein after dilution with a compatible admixture diluent selected from 5% dextrose injection, 0.9% sodium chloride injection, or lactated Ringer's injection, the diluted composition remains clear and free from precipitation for at least 17 days at 25 °C and at 2-8 °C.

30. The injectable composition according to claim 14, wherein the combination of ethanol, present in 60-65% w/v, and PEG 35 castor oil, present in 1.0-1.5% w/v, provides physical stability of metolazone such that precipitation is not observed for at least 17 days after dilution with a compatible admixture diluent.
